SEQUENCE LISTING (10 sequences)

SEQ ID NO: 1 - DNA, 1355 bp, Agrobacterium sp.
  source 1..1355: mol_type = genomic DNA
atagcagtcgacgccccgcaggggagtggcagacgggtgagtaacgcgtgggaacataccctttcctgcggaatagctccgggaaactggaattaataccgcatacgccctacgggggaaagatttatcggggaaggattggcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatatttaagtcaggggtgaaatcccagagctcaactctggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtttgggcagtggagacattgtccttcagttaggctgggcccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctatcca

SEQ ID NO: 2 - DNA, 1388 bp, Pantoea sp.
  source 1..1388: mol_type = genomic DNA
ccatgcagtcggacggtagcacagataagcttgctccttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccatatgggattatctagtaggcggggtaatggcccacctaggcgacgatccctagctggtctgagaggatgaccagccacactggaactgagacacggtccagactcctacgggaggcagcagtggggaatattgcacaatgggcgcaagcctgatgcacccatgccgcgtgtatgaagaaggccttcgggttgtaaagtactttcagcggggaggaaggcgacggggttaataaccctgtcgattgacgttacccgcagaagacgcaccggctaactccgtgccagcagccgcggtaatacggagggtgcaagcgttaatcggaattactgggcgtaaagcgcacgcaggcggtctgttaagtcacatgtgaaatcccccgggcttaacctgggaactgcatttgaaactggcaggcttgagtcttgtagaggggggtagaattcctggtgtagcggtgaaatgcgtagagatctggacgaataccggtggcgatcgcggccccctggacaaagactgacgctcacgtgcgaaagcgtggggagcaaactcgattacataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaatttagcagagatgctttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtagtt

SEQ ID NO: 3 - DNA, 1313 bp, Sphingobium sp.
  source 1..1313: mol_type = genomic DNA
catgcagtcgacgagcctttcggggctagtggcgcacgggtgcgtaacgcgtgggaatctgcccttgggttcggaataacgtcgggaaactgacgctaataccggatgatgacgtaagtccaaagatttatcgcccagggatgagcccgcgtaggattagctagttggtgaggtaaaggctcaccaaggcgacgatccttagctggtctgagaggatgatcagccacactgggactgagacacggcccagactcctacgggaggcagcagtagggaatattggacaatgggggcaaccctgatccagcaatgccgcgtgagtgatgaaggccttagggttgtaaagctcttttacccgggatgataatgaatacggagggggctagcgttgttcggaattactgggcgtaaagcgcacgtaggcggcgatttaagtcagaggtgaaagcccggggctcaacccccggaatagcctttgagactggattgcttgaacatcggagaggtgagtggaattccgagtgtagaggtgaaatttcgtagatattcggaagaacaccagtggcgaaggcggctcactggacgattgttgacgctgaggtgcgaaagcgtggggagcaaacaggattagatacccctggtagtccacgccgtaaacgatgataactagctgctggggctcatggagtttcggtggcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagaatacttgggttaagtcccgcaacgagcgcaaccctcgcctttagttgccatcatttagttgggtactctaaaggaaccgccggtgataagccggaggaaggtggggatgacgtcaagtcctcatggcccttacgcgttgggctacacacgtgctacaatggcgactacagtgggcagccactccgcgaggaggagctaatctccaaaagtcgtctcagttcggattgttctctgcaactcgagagcatgaaggcggaatcgctagtaatcgcggatcagcatgccgcggtgaatacgttcccaggccttgtacacaccgcccgtcacaccatgggagttggtttcacccgaaggctgtgcgctaaccgcaaggaggcagcagacca

SEQ ID NO: 4 - DNA, 1422 bp, Pseudomonas sp.
  source 1..1422: mol_type = genomic DNA
acacatgcagtcgagcggtagagaggtgcttgcacctcttgagagcggcggacgggtgagtaaagcctaggaatctgcctggtagtgggggataacgctcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcattcaaaactgtcgagctagagtatggtagagggtggtggaatttcctgttgtagcggtgaaatgcgtgatacagatataggaaggaacaccagtggcgaaggcgacccacctggactgatactgacactgaggtgcgaaagcgtggggagcaaacaggattagataccctgggtagtccacgcccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagtaccttaccaggccttgacatcctatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggtaccacg

SEQ ID NO: 5 - DNA, 1352 bp, Enterobacter sp.
  source 1..1352: mol_type = genomic DNA
agtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaagggcggccccctggacaaagactgacgctcaggtgcgaaagcgtggggagcaaacaggattagatacccctggtagtccacgccgtaaacgatgtcgacttggaggttgtgcccttgaggcgtggcttccggagctaacgcgttaagtcgaccgcctggggagtacggccgcaaggataaaaccttaatgaattgacgggggcccgcacaagcggtggagcatgtggtttaattcgatgcaacgcgaagaacctttgctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgaactcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgctac

SEQ ID NO: 6 - DNA, 1394 bp, Micrococcus sp.
  source 1..1394: mol_type = genomic DNA
gcatgcagtcgacgatgaagcccagcttgctgggtggattagtggcgaacgggtgagtaacacgtgagtaacctgcccttaactctgggataagcctgggaaactgggtctaataccggatatgagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagtagcttaccaaggcttgacatgtactcgatcggcgtagagatacggtttcccgttaggggcgggttctgtggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtac

SEQ ID NO: 7 - DNA, 1421 bp, Bacillus sp.
  source 1..1421: mol_type = genomic DNA
atgcagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggtttacgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaatttccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaacgatcccgttctaccttaccaggtgatgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtgaggtaaccttcatggagccagccgcc

SEQ ID NO: 8 - DNA, 1419 bp, Pantoea sp.
  source 1..1419: mol_type = genomic DNA
ccatgcagtcggacggtagcacagaggagcttgctcctcgggtgacgagtggcggacgggtgagtaatgtctggggatctgcccgatagagggggataaccactggaaacggtggctaataccgcaaaacgtcgcaagaccaaagagggggaccttcgggcctctcactatcggatgaacccagatgggattagctagtaggcggggtaacggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgtggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaatttccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggattaaactcaaatgaattgacgggggcccgcacaagcggtggagcatgtggtttaattcgatgcaacgcgaagaaccatacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggctagcttaaccttcgggagggcgctaccactt

SEQ ID NO: 9 - DNA, 1420 bp, Acinetobacter sp.
  source 1..1420: mol_type = genomic DNA
cacatgcagtcgagcggggagagtagcttgctacttgacctagcggcggacgggtgagtaatgcttaggaatctgcctattagtgggggacaacatctcgaaagggatgctaataccgcatacgtcctacgggagaaagcaggggaccttcgggccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagcgaggaggaggctaccgagattaatactcttggatagtggacgttactcgcagaataagcaccggctaactctgtgccagcagccgcggtaatacagagggtgcaagcgttaatcggatttactgggcgtaaagcgcgcgtaggtggccaattaagtcaaatgtgaaatccccgagcttaacttgggaattgcattcgatactggttggctagagtatgggagaggatggtagaattccaggtgtagcggtgaaatgcgtagagatctggaggaataccgatggcgaaggcagccatctggcctaatactgacactgaggtgcgaaagcatggggagcaaacaggattagataccctggtagtccatgccgtaaacgatgtctactagccgttggggcctttgctggctttagtggcgcagctaacgcgataagtagaccgcctggggagtacggtcgcaagactaaaactcaaatgaattgacgggggcccgcacaagcggtggagcatgtggtttaattcgatgcaacgcgaagtagcttacctggtcttgacatagtatcttctttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctcgcgagaggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtagggtaggtccttaacgtctaagggaggacgctaccacgg

SEQ ID NO: 10 - DNA, 1436 bp, Paenibacillus sp.
  source 1..1436: mol_type = genomic DNA
atacatgcagtcgagcggacttgcatgagaagcttgcttctctgatggttagcggcggacgggtgagtaacacgtaggcacctgccctcaagcttgggacaactaccggaaacggtagctaataccgaatagttgttttcttctcctgaagaaaactggaaagacggagcaatctgtcacttggggatgggcctgcggcgcattagctagttggtggggtaacggctcaccaaggcgacgatgcgtagccgacctgagagggtgatcggccacactgggactgagacacggcccagactcctacgggaggcagcagtagggaatcttccgcaatgggcgaaagcctgacggagcaatgccgcgtgagtgatgaaggttttcggatcgtaaagctctgttgccagggaagaacgcttgggagagtaactgctctcaaggtgacggtacctgagaagaaagccccggctaactacgtgccagcagccgcggtaatacgtagggggcaagcgttgtccggaattattgggcgtaaagcgcgcgcaggcggtcatttaagtctggtgtttaatcccggggctcaaccccggatcgcactggaaactgggtgacttgagtgcagaagaggagagtggaattccacgtgtagcggtgaaatgcgtagatatgtggaggaacaccagtggcgaagcgcgactctctgggctgtaactgacgctgaggcgcgaaagcgtggggagcaaacaggattagataccctggtagtccacgccgtaaacgatgagtgctaggtgttaggggtttcgatacccttggtgccgaagttaacacattaagcactccgcctggggagtacggtcgcaagactgaaactcaaaggaattgacggggacccgcacaagcagtggagtatgtggttttattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttgatcttagttgccagcacttcgggtgggcactctaaggtgactgccggtgacaaaccggaggaaggtggggatgacgtcaaatcatcatgccccttatgacctgggctacacacgtactacaatggccggtacaacgggctgtgaagccgcgaggtggaacgaatcctaaaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaaggtgatc

(19) United States
(12) Patent Application Publication
(10) Pub. No.: US 2025/0314631 A1
(43) Pub. Date: Oct. 9, 2025

(54) PLANT-ENDOPHYTE COMBINATIONS AND USES THEREFOR

(71) Applicants: AIT AUSTRIAN INSTITUTE OF TECHNOLOGY GMBH, Vienna (AT); INDIGO AG, INC., Boston, MA (US)

(72) Inventors: Birgit Mitter, Giesshübl (AT); Milica PASTAR, Wien (AT); Angela SESSITSCH, Vienna (AT); Karen AMBROSE, Cambridge, MA (US); Xuecheng ZHANG, Brookline, MA (US); Guenther REICHENBERGER, Vienna (AT); Nikolaus PFAFFENBICHLER, Vienna (AT)

(21) Appl. No.: 18/957,191

(22) Filed: Nov. 22, 2024

Related U.S. Application Data
(63) Continuation of application No. 18/092,534, filed on Jan. 3, 2023, now abandoned.
Application No. 18/092,534 is a continuation of application No. 16/212,559, filed on Dec. 6, 2018, now abandoned.
Application No. 16/212,559 is a continuation of application No. 15/321,595, filed on Dec. 22, 2016, now abandoned.

(30) Foreign Application Priority Data
Dec. 24, 2014 (WO) PCT/US2014/072399
Dec. 24, 2014 (WO) PCT/US2014/072400

(51) Int. Cl. G01N 33/00; A01N 63/25; C12N 1/20

(52) U.S. Cl. CPC G01N 33/0098; A01N 63/25; C12N 1/20

(57) ABSTRACT

The disclosure provides materials and methods for conferring improved plant traits or benefits on plants. The materials can include a formulation comprising an exogenous endophytic bacterial population, which can be disposed on an exterior surface of a seed or seedling, typically in an amount effective to colonize the plant. The formulations can include at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

### CROSS REFERENCE TO RELATED APPLICATIONS

This application is a continuation of U.S. application Ser. No. 18/092,534, filed Jan. 3, 2023, which is a continuation of U.S. application Ser. No. 16/212,559, filed Dec. 6, 2018, which is a continuation of U.S. application Ser. No. 15/321,595, filed Dec. 22, 2016, which is the National Stage of International Application No. PCT/US2015/038110, filed Jun. 26, 2015, which claims the benefit of and priority to U.S. application Ser. No. 14/315,804, filed Jun. 26, 2014, and International Application No. PCT/US2014/072399, filed Dec. 24, 2014, and International Application No. PCT/US2014/072400, filed Dec. 24, 2014, which are incorporated in their entirety by reference for all purposes.

### SEQUENCE LISTING

The instant application is being filed along with a Sequence Listing in electronic format. The Sequence Listing file, entitled INDIGO-60001PCTUS4_SL.xml, was created on Nov. 11, 2024, and is 23,419 bytes in size. The information in electronic format of the Sequence Listing is incorporated herein by reference in its entirety.

### BACKGROUND

With limited arable land coupled with rising demand of a steadily increasing human population that could reach 9 billion by 2050, food supply is a global challenge making production of economically attractive and high quality food, free from unacceptable levels of agrochemicals, a dire need.

Traditional plant breeding strategies to enhance plant traits are relatively slow and inefficient. For example, breeding plants for increased tolerance to abiotic stress requires abiotic stress-tolerant founder lines for crossing with other germplasm to develop new abiotic stress-resistant lines. Limited germplasm resources for such founder lines and incompatibility in crosses between distantly related plant species represent significant problems encountered in conventional breeding. Breeding for stress tolerance has often been inadequate given the incidence of stresses and the impact that stresses have on crop yields in most environments of the world.

### SUMMARY OF THE INVENTION

The present invention is based on the systematic efforts to discover endophytic bacterial species that have the potential to greatly improve agricultural productivity. The endophytic bacterial strains extensively characterized herein are able to confer onto the host plant several key fitness benefits and, as such, offer the possibility of improving yields of agricultural crops without the need for time-consuming breeding efforts or genetic modification.

In a first aspect, the present invention provides for an agricultural plant or portion thereof comprising an exogenous endophytic bacterial population disposed on an exterior surface of the seed or seedling in an amount effective to colonize the plant, and further comprising a formulation that comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient. The agricultural plant can be a mature plant. In other cases, it can be a seedling. In still other cases, it can be a seed of an agricultural plant. In one particular embodiment, the agricultural plant is a seed or seedling.

In one embodiment, the endophytic bacterial population consists essentially of an endophytic bacterium comprising a 16S rRNA nucleic acid sequence at least 95%, at least 96%, at least 97%, at least 98%, at least 99%, or at least 99.5% identical to a nucleic acid sequence selected from the group consisting of SEQ ID NOs: 1-10.

In one particular embodiment, the endophytic bacterium is a species of Agrobacterium, family Rhizobiaceae. In a particular embodiment, the Agrobacterium species is identified on the basis of its rDNA sequence, as outlined herein. In a particular embodiment, the Agrobacterium species comprises a 16S rDNA sequence that is at least 95% identical to SEQ ID NO: 1. In another embodiment, the Agrobacterium species comprises a 16S rDNA sequence that is at least 99% identical to SEQ ID NO: 1. In still another embodiment, the Agrobacterium species comprises a 16S rDNA sequence that is identical to SEQ ID NO: 1. In still another embodiment, the Agrobacterium species is the isolate FA13.

In another embodiment, the endophytic bacterium is a species of Pantoea, family Rhizobiaceae. In a particular embodiment, the Pantoea species is identified on the basis of its rDNA sequence, as outlined herein. In a particular embodiment, the Pantoea species comprises a 16S rDNA sequence that is at least 95% identical to SEQ ID NO: 2. In another embodiment, the Pantoea species comprises a 16S rDNA sequence that is at least 99% identical to SEQ ID NO: 2. In still another embodiment, the Pantoea species comprises a 16S rDNA sequence that is identical to SEQ ID NO: 2. In still another embodiment, the Pantoea species is the isolate FF34.

In another embodiment, the endophytic bacterium is a species of Sphingobium, family Rhizobiaceae. In a particular embodiment, the Sphingobium species is identified on the basis of its rDNA sequence, as outlined herein. In a particular embodiment, the Sphingobium species comprises a 16S rDNA sequence that is at least 95% identical to SEQ ID NO: 3. In another embodiment, the Sphingobium species comprises a 16S rDNA sequence that is at least 99% identical to SEQ ID NO: 3. In still another embodiment, the Sphingobium species comprises a 16S rDNA sequence that is identical to SEQ ID NO: 3. In still another embodiment, the Sphingobium species is the isolate FC42.

In another embodiment, the endophytic bacterium is a species of Pseudomonas, family Pseudomonadaceae. In a particular embodiment, the Pseudomonas species is identified on the basis of its rDNA sequence, as outlined herein. In a particular embodiment, the Pseudomonas species comprises a 16S rDNA sequence that is at least 95% identical to SEQ ID NO: 4. In another embodiment, the Pseudomonas species comprises a 16S rDNA sequence that is at least 99% identical to SEQ ID NO: 4. In still another embodiment, the Pseudomonas species comprises a 16S rDNA sequence that is identical to SEQ ID NO: 4. In still another embodiment, the Pseudomonas species is the isolate FB12.

In another embodiment, the endophytic bacterium is a species of Enterobacter, family Enterobacteriaceae. In a particular embodiment, the Enterobacter species is identified on the basis of its rDNA sequence, as outlined herein. In a particular embodiment, the Enterobacter species comprises a 16S rDNA sequence that is at least 95% identical to SEQ ID NO: 5. In another embodiment, the Enterobacter species comprises a 16S rDNA sequence that is at least 99% identical to SEQ ID NO: 5. In still another embodiment, the Enterobacter species comprises a 16S rDNA sequence that is identical to SEQ ID NO: 5. In still another embodiment, the Enterobacter species is the isolate FD17.

In another embodiment, the endophytic bacterium is a species of Micrococcus, family Micrococcaceae. In a particular embodiment, the Micrococcus species is identified on the basis of its rDNA sequence, as outlined herein. In a particular embodiment, the Micrococcus species comprises a 16S rDNA sequence that is at least 95% identical to SEQ ID NO: 6. In another embodiment, the Micrococcus species comprises a 16S rDNA sequence that is at least 99% identical to SEQ ID NO: 6. In still another embodiment, the Micrococcus species comprises a 16S rDNA sequence that is identical to SEQ ID NO: 6. In still another embodiment, the Micrococcus species is the isolate S2.

In another embodiment, the endophytic bacterium is a species of Bacillus, family Bacillaceae. In a particular embodiment, the Bacillus species is identified on the basis of its rDNA sequence, as outlined herein. In a particular embodiment, the Bacillus species comprises a 16S rDNA sequence that is at least 95% identical to SEQ ID NO: 7. In another embodiment, the Bacillus species comprises a 16S rDNA sequence that is at least 99% identical to SEQ ID NO: 7. In still another embodiment, the Bacillus species comprises a 16S rDNA sequence that is identical to SEQ ID NO: 7. In still another embodiment, the Bacillus species is the isolate S4.

In another embodiment, the endophytic bacterium is a species of Pantoea, family Enterobacteriaceae. In a particular embodiment, the Pantoea species is identified on the basis of its rDNA sequence, as outlined herein. In a particular embodiment, the Pantoea species comprises a 16S rDNA sequence that is at least 95% identical to SEQ ID NO: 8. In another embodiment, the Pantoea species comprises a 16S rDNA sequence that is at least 99% identical to SEQ ID NO: 8. In still another embodiment, the Pantoea species comprises a 16S rDNA sequence that is identical to SEQ ID NO: 8. In still another embodiment, the Pantoea species is the isolate S6.

In another embodiment, the endophytic bacterium is a species of Acinetobacter, family Moraxellaceae. In a particular embodiment, the Acinetobacter species is identified on the basis of its rDNA sequence, as outlined herein. In a particular embodiment, the Acinetobacter species comprises a 16S rDNA sequence that is at least 95% identical to SEQ ID NO: 9. In another embodiment, the Acinetobacter species comprises a 16S rDNA sequence that is at least 99% identical to SEQ ID NO: 9. In still another embodiment, the Acinetobacter species comprises a 16S rDNA sequence that is identical to SEQ ID NO: 9. In still another embodiment, the Acinetobacter species is the isolate S9.

In another embodiment, the endophytic bacterium is a species of Paenibacillus, family Paenibacillaceae. In a particular embodiment, the Paenibacillus species is identified on the basis of its rDNA sequence, as outlined herein. In a particular embodiment, the Paenibacillus species comprises a 16S rDNA sequence that is at least 95% identical to SEQ ID NO: 10. In another embodiment, the Paenibacillus species comprises a 16S rDNA sequence that is at least 99% identical to SEQ ID NO: 10. In still another embodiment, the Paenibacillus species comprises a 16S rDNA sequence that is identical to SEQ ID NO: 10. In still another embodiment, the Paenibacillus species is the isolate S10.

In certain cases, the endophytic bacterial population is disposed in an amount effective to be detectable within a target tissue of the mature agricultural plant selected from a fruit, a seed, a leaf, or a root, or portion thereof.

In certain embodiments, the seed or seedling comprises at least about 100 CFU, for example, at least about 200 CFU, at least about 300 CFU, at least about 500 CFU, at least about 1,000 CFU, at least about 3,000 CFU, at least about 10,000 CFU, at least about 30,000 CFU, at least about 100,000 CFU, at least about 10≢CFU, or more, of the endophytic bacterial population on its exterior surface.

In another embodiment, the endophytic bacterial population is disposed on an exterior surface or within a tissue of the seed or seedling in an amount effective to be detectable in an amount of at least about 100 CFU, for example, at least about 200 CFU, at least about 300 CFU, at least about 500 CFU, at least about 1,000 CFU, at least about 3,000 CFU, at least about 10,000 CFU, at least about 30,000 CFU, at least about 100,000 CFU or more per gram fresh weight of the mature agricultural plant.

In another embodiment, the endophytic bacterial population is disposed on the surface or within a tissue of the seed or seedling in an amount effective to increase the biomass of the fruit or cob from the resulting plant by at least 1%, at least 2%, at least 3%, at least 4%, at least 5%, at least 6%, at least 7%, at least 8%, at least 9%, or at least 10% when compared with a reference agricultural plant.

In still another embodiment, the endophytic bacterial population is disposed on the surface or within a tissue of the seed or seedling in an amount effective to detectably colonize the soil environment surrounding the mature agricultural plant when compared with a reference agricultural plant.

In some cases, the endophytic bacterial population is disposed in an amount effective to increase root biomass by at least 1%, at least 2%, at least 3%, at least 4%, at least 5%, at least 6%, at least 7%, at least 8%, at least 9%, or at least 10% when compared with a reference agricultural plant.

In some embodiments, the endophytic bacterial population is disposed on the surface or within a tissue of the seed or seedling in an amount effective to increase the rate of seed germination when compared with a reference agricultural plant.

In another embodiment, the endophytic bacterial population is disposed on the surface or within a tissue of the seed or seedling in an amount effective to detectably induce production of auxin in the seed or seedling.

In one embodiment, the endophytic bacterial population is cultured prior to disposition on the seed or seedling. In one embodiment, the endophytic bacterial population is cultured in a synthetic or semi-synthetic medium prior to disposition on the seed or seedling.

In certain cases, the endophytic bacterial population can be modified. In one embodiment, the endophytic bacterial population is genetically modified. In another embodiment, the endophytic bacterial population is modified such that it has enhanced compatibility with an antimicrobial agent when compared with an unmodified control. The antimicrobial agent is an antibacterial agent. Alternatively, the antimicrobial agent can be an antifungal agent. In some cases, the modified endophytic bacterial population exhibits at least 3 fold greater, for example, at least 5 fold greater, at least 10 fold greater, at least 20 fold greater, at least 30 fold greater or more resistance to the antimicrobial agent when compared with an unmodified control. In one embodiment, the antimicrobial agent is glyphosate.

The seed or seedling of the agricultural plant can be a monocot. For example, it can be a corn seed or seedling. Alternatively, it can be a wheat seed or seedling. In other embodiments, it can be a barley seed or seedling. In still other cases, it can be a rice seed or seedling.

In another embodiment, the seed or seedling is a dicot. For example, it can be a cotton seed or seedling, a soy seed or seedling, or a tomato seed or seedling.

In still another embodiment, the seed or seedling can be derived from a transgenic plant. In another embodiment, the seed or seedling can be a hybrid seed or seedling.

In one particular embodiment, the seed is a corn seed, and further comprises at least about 10,000 CFU of the endophytic bacterial population consisting essentially of an endophytic bacterium comprising a 16S rRNA nucleic acid sequence that is at least 95%, 96%, 97%, for example, at least 98%, at least 99%, at least 99.5%, or 100% identical to a nucleic acid sequence selected from the group consisting of SEQ ID NOs: 1-10 disposed on the exterior surface of the seed, and further comprising a formulation comprising a microbial stabilizer.

In another aspect, the invention provides for a substantially uniform population of seeds comprising a plurality of seeds described above. Substantial uniformity can be determined in many ways. In some cases, at least 10%, for example, at least 20%, at least 30%, at least 40%, at least 50%, at least 60%, at least 70%, at least 75%, at least 80%, at least 90%, at least 95% or more of the seeds in the population, contains the endophytic bacterial population in an amount effective to colonize the plant disposed on the surface of the seeds. In other cases, at least 10%, for example, at least 20%, at least 30%, at least 40%, at least 50%, at least 60%, at least 70%, at least 75%, at least 80%, at least 90%, at least 95% or more of the seeds in the population, contains at least 100 CFU on its surface, for example, at least 200 CFU, at least 300 CFU, at least 1,000 CFU, at least 3,000 CFU, at least 10,000 CFU, at least 30,000 CFU, at least 100,000 CFU, at least 300,000 CFU, or at least 1,000,000 CFU per seed or more.

In yet another aspect, the present invention provides for a bag comprising at least 1,000 seeds as described herein above. The bag further comprises a label describing the seeds and/or said endophytic bacterial population.

In still another aspect of the present invention, a plant or part or tissue of the plant, or progeny thereof is disclosed, which is generated by growing the seed or seedling described herein above.

In yet another aspect, disclosed are substantially uniform populations of plants produced by growing a plurality of seeds, seedlings, or progeny thereof. In some cases, at least 75%, at least 80%, at least 90%, at least 95% or more of the plants in the population comprise an amount of the endophytic bacterial population effective to increase the root biomass of the plant by at least 1%, at least 2%, at least 3%, at least 4%, at least 5%, at least 6%, at least 7%, at least 8%, at least 9%, or at least 10%. In other cases, at least 10%, for example at least 20%, at least 30%, at least 40%, at least 50%, at least 60%, at least 70%, at least 75%, at least 80%, at least 90%, at least 95% or more of the plants comprise a microbe population that is substantially similar.

In yet another aspect of the present invention, disclosed is an agricultural field comprising the population described above. The field generally comprises at least 100 plants, for example, at least 1,000 plants, at least 3,000 plants, at least 10,000 plants, at least 30,000 plants, at least 100,000 plants or more in the field. In certain cases, the population of plants occupies at least about 100 square feet of space, and at least about 10%, for example, at least 20%, at least 30%, at least 40%, at least 50%, at least 60%, at least 70%, at least 80%, at least 90% or more than 90% of the population comprises an amount of the endophytic bacterial population effective to increase the root biomass of the plant by at least 1%, at least 2%, at least 3%, at least 4%, at least 5%, at least 6%, at least 7%, at least 8%, at least 9%, or at least 10%. In another embodiment, the population of plants occupies at least about 100 square feet of space, wherein and at least about 10%, for example, at least 20%, at least 30%, at least 40%, at least 50%, at least 60%, at least 70%, at least 80%, at least 90% or more than 90% of the population comprises the microbe in reproductive tissue. In another embodiment, the population of plants occupies at least about 100 square feet of space, and at least about 10%, for example, at least 20%, at least 30%, at least 40%, at least 50%, at least 60%, at least 70%, at least 80%, at least 90% or more than 90% of the population comprises at least 100 CFUs, 1,000 CFUs, 10,000 CFUs, 100,000 CFUs or more of the endophytic bacterial population.

In another aspect of the invention, provided are preparations comprising a population of endophytic bacteria described herein and further comprising at least one agent selected from the group consisting of an agriculturally acceptable carrier, a tackifier, a microbial stabilizer, a fungicide, an antibacterial agent, an herbicide, a nematicide, an insecticide, a plant growth regulator, a rodenticide, and a nutrient, and wherein the population comprises an amount of endophytes sufficient to improve an agronomic trait of the population of seeds. In one embodiment, the endophytic bacterial population consists essentially of an endophytic bacterium comprising a 16S rRNA nucleic acid sequence at least 95% identical to a nucleic acid sequence selected from the group consisting of SEQ ID NOs: 1-10.

In one embodiment, the preparation is substantially stable at temperatures between about 4° C. and about 45° C. for at least about seven days.

In another embodiment, the preparation is formulated to provide at least 100 endophytes per seed, for example, at least 300 endophytes, at least 1,000 endophytes, at least 3,000 endophytes, at least 10,000 endophytes, at least 30,000 endophytes, at least 100,000 endophytes, at least 300,000 endophytes, or at least 1,000,000 endophytes per seed.

In another embodiment, the preparation is formulated to provide a population of plants that demonstrates a substantially homogenous growth rate when introduced into agricultural production.

In still another aspect, the present invention provides for a method of producing a commodity plant product. The method generally comprises obtaining a plant or plant tissue from the agricultural plant comprising the endophytic bacteria as described herein above, and producing the commodity plant product therefrom. In certain cases, the commodity plant product is selected from the group consisting of grain, flour, starch, seed oil, syrup, meal, flour, oil, film, packaging, nutraceutical product, an animal feed, a fish fodder, a cereal product, a processed human-food product, a sugar or an alcohol and protein.

In a related aspect, the present invention provides for a commodity plant product comprising a plant or part thereof and further comprising the endophytic bacterial population or a portion thereof in a detectable level.

In yet another aspect of the present invention, provided is a method for preparing an agricultural plant or a portion thereof comprising an endophytic bacterial population. The method generally comprises applying to the seed or seedling a formulation comprising an endophytic bacterial population consisting essentially of an endophytic bacterium comprising a 16S rRNA nucleic acid sequence at least 95% identical, for example, at least 96%, at least 97%, at least 98% identical, at least 99% identical, at least 99.5% identical, or 100% identical to a nucleic acid sequence selected from the group consisting of SEQ ID NOs: 1-10. In one embodiment, the formulation further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient. In some cases, the agricultural plant can be a seedling.

In other cases, the agricultural plant can be a seed. In a particular embodiment, the agricultural plant is a seed or a seedling. In another embodiment, the method further comprises applying at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

In a final aspect, the present invention provides for a method for conferring one or more fitness benefits to an agricultural plant. The method generally comprises providing an agricultural plant or portion thereof, contacting said plant or portion thereof with a formulation comprising an exogenous endophytic bacterial population consisting essentially of an endophytic bacterium comprising a 16S rRNA nucleic acid sequence at least 95% identical, for example, at at least 96%, at least 97%, least 98% identical, at least 99% identical, at least 99.5% identical, or 100% identical to a nucleic acid sequence selected from the group consisting of SEQ ID NOs: 1-10, disposed on an exterior surface in an amount effective to colonize the mature plant, wherein the formulation further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient, and allowing the seed or seedling to grow under conditions that allow the endophytic bacterium to colonize the plant. In some cases, the agricultural plant can be a seedling. In other cases, the agricultural plant can be a seed. In a particular embodiment, the agricultural plant is a seed or a seedling.

In one embodiment, the one or more of the fitness benefits are selected from the group consisting of increased germination, increased biomass, increased flowering time, increased biomass of the fruit or grain, increased grain or fruit yield, and increased drought tolerance.

### DETAILED DESCRIPTION

### Brief Description of the Figures

FIG. 1A show the increases in root biomass in maize plants inoculated with the bacterial endophyte populations when compared with uninoculated control plants.

FIG. 1B show the increases in shoot biomass in maize plants inoculated with the bacterial endophyte populations when compared with uninoculated control plants.

FIG. 1C show the increases in total biomass in maize plants inoculated with the bacterial endophyte populations when compared with uninoculated control plants.

FIG. 2 shows the increases in stomatal conductance in maize plants inoculated with the bacterial endophyte populations when compared with uninoculated control plants.

FIG. 3 shows the increase in photosynthetic rates in maize plants inoculated with the bacterial endophyte populations when compared with uninoculated control plants.

FIG. 4 shows the increases in PS II photochemical efficiency (Fv/Fm) in maize plants inoculated with the bacterial endophyte populations, when compared with uninoculated control plants.

FIG. 5 shows the increases in leaf area in maize plants inoculated with the bacterial endophyte populations, when compared with uninoculated control plants.

FIG. 6 shows the increases in chlorophyll content in maize plants inoculated with the bacterial endophyte populations, when compared with uninoculated control plants.

### DEFINITIONS

A “synthetic combination” includes a combination of a host plant and an endophyte. The combination may be achieved, for example, by coating the surface of the seed of a plant, such as an agricultural plant, or host plant tissues with an endophyte.

As used herein, an “agricultural seed” is a seed used to grow a plant in agriculture (an “agricultural plant”). The seed may be of a monocot or dicot plant, and is planted for the production of an agricultural product, for example grain, food, fiber, etc. As used herein, an agricultural seed is a seed that is prepared for planting, for example, in farms for growing.

An “endophyte”, or “endophytic microbe” includes an organism capable of living within a plant or associated therewith. An endophyte may refer to a bacterial or fungal organism that may confer an increase in yield, biomass, resistance, or fitness in its host plant. Endophytes may occupy the intracellular or extracellular spaces of plant tissue, including the leaves, stems, flowers, fruits, seeds, or roots. An endophyte can be a fungus, or a bacterium. As used herein, the term “microbe” is sometimes used to describe an endophyte.

In some embodiments, the invention contemplates the use of microbes that are “exogenous” to a seed or plant. As used herein, a microbe is considered exogenous to the seed or plant if the seed or seedling that is unmodified (e.g., a seed or seedling that is not treated with the endophytic bacterial population descried herein) does not contain the microbe.

In other cases, the invention contemplates the synthetic combinations of agricultural plants and an endophytic microbe population, in which the microbe population is “heterologously disposed” on the surface of or within a tissue of the agricultural plant. As used herein, a microbe is considered “heterologously disposed” on the surface or within a plant (or tissue) when the microbe is applied or disposed on the plant in a number or within a tissue in a number that is not found on that plant prior to application of the microbe. As such, a microbe is deemed heterologously disposed when applied on the plant that either does not naturally have the microbe on its surface or within the particular tissue to which the microbe is disposed, or does not naturally have the microbe on its surface or within the particular tissue in the number that is being applied. For the avoidance of doubt, “heterologously disposed” contemplates use of microbes that are “exogenous” to a seed or plant.

In some cases, the present invention contemplates the use of microbes that are “compatible” with agricultural chemicals for example, a fungicide, an anti-bacterial compound, or any other agent widely used in agricultural which has the effect of interfering with optimal growth of microbes. As used herein, a microbe is “compatible” with an agricultural chemical, when the microbe is modified or otherwise adapted to grow in, or otherwise survive, the concentration of the agricultural chemical used in agriculture. For example, a microbe disposed on the surface of a seed is compatible with the fungicide metalaxyl if it is able to survive the concentrations that are applied on the seed surface.

“Biomass” means the total mass or weight (fresh or dry), at a given time, of a plant tissue, plant tissues, an entire plant, or population of plants, usually given as weight per unit area. The term may also refer to all the plants or species in the community (community biomass).

Some of the compositions and methods described herein involve endophytic microbes in an amount effective to colonize a plant. As used herein, a microbe is said to “colonize” a plant or seed when it can exist in an endophytic relationship with the plant in the plant environment, for example inside the plant or a part or tissue thereof, including the seed.

Some compositions described herein contemplate the use of an agriculturally compatible carrier. As used herein an “agriculturally compatible carrier” is intended to refer to any material, other than water, which can be added to a seed or a seedling without causing/having an adverse effect on the seed, the plant that grows from the seed, seed germination, or the like.

A “transgenic plant” includes a plant or progeny plant of any subsequent generation derived therefrom, wherein the DNA of the plant or progeny thereof contains an introduced exogenous DNA segment not naturally present in a non-transgenic plant of the same strain.

The transgenic plant may additionally contain sequences that are native to the plant being transformed, but wherein the “exogenous” gene has been altered in order to alter the level or pattern of expression of the gene, for example, by use of one or more heterologous regulatory or other elements.

As used herein, a nucleic acid has “homology” or is “homologous” to a second nucleic acid if the nucleic acid sequence has a similar sequence to the second nucleic acid sequence. The terms “identity”, “percent sequence identity” or “identical” in the context of nucleic acid sequences refer to the residues in the two sequences that are the same when aligned for maximum correspondence. There are a number of different algorithms known in the art that can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, or at least about 90%, or at least about 95%, 96%, 97%, 98% or 99% of the nucleotide bases, as measured by any well-known algorithm of sequence identity, such as FASTA, BLAST or Gap, as discussed above.

The present invention is directed to methods and compositions of bacterial endophytes, and plant-endophyte combinations that confer a fitness benefit in agricultural plants.

### Bacterial Endophyte

In a first aspect, disclosed is a composition comprising a pure culture of a bacterial endophyte.

In one embodiment, the endophytic bacterium is a species of Agrobacterium. In a particular embodiment, the Agrobacterium species is identified on the basis of its rDNA sequence, as outlined herein. In a particular embodiment, the Agrobacterium species comprises a 16S rDNA sequence that is at least 95% identical to SEQ ID NO: 1. In another embodiment, the Agrobacterium species comprises a 16S rDNA sequence that is at least 99% identical to SEQ ID NO: 1. In still another embodiment, the Agrobacterium species comprises a 16S rDNA sequence that is identical to SEQ ID NO: 1. In still another embodiment, the Agrobacterium species is the isolate FA13.

In another embodiment, the endophytic bacterium is a species of Pantoea. In a particular embodiment, the Pantoea species is identified on the basis of its rDNA sequence, as outlined herein. In a particular embodiment, the Pantoea species comprises a 16S rDNA sequence that is at least 95% identical to SEQ ID NO: 2. In another embodiment, the Pantoea species comprises a 16S rDNA sequence that is at least 99% identical to SEQ ID NO: 2. In still another embodiment, the Pantoea species comprises a 16S rDNA sequence that is identical to SEQ ID NO: 2. In still another embodiment, the Pantoea species is the isolate FF34.

In another embodiment, the endophytic bacterium is a species of Sphingobium. In a particular embodiment, the Sphingobium species is identified on the basis of its rDNA sequence, as outlined herein. In a particular embodiment, the Sphingobium species comprises a 16S rDNA sequence that is at least 95% identical to SEQ ID NO: 3. In another embodiment, the Sphingobium species comprises a 16S rDNA sequence that is at least 99% identical to SEQ ID NO: 3. In still another embodiment, the Sphingobium species comprises a 16S rDNA sequence that is identical to SEQ ID NO: 3. In still another embodiment, the Sphingobium species is the isolate FC42.

In another embodiment, the endophytic bacterium is a species of Pseudomonas. In a particular embodiment, the Pseudomonas species is identified on the basis of its rDNA sequence, as outlined herein. In a particular embodiment, the Pseudomonas species comprises a 16S rDNA sequence that is at least 95% identical to SEQ ID NO: 4. In another embodiment, the Pseudomonas species comprises a 16S rDNA sequence that is at least 99% identical to SEQ ID NO: 4. In still another embodiment, the Pseudomonas species comprises a 16S rDNA sequence that is identical to SEQ ID NO: 4. In still another embodiment, the Pseudomonas species is the isolate FB12.

In another embodiment, the endophytic bacterium is a species of Enterobacter. In a particular embodiment, the Enterobacter species is identified on the basis of its rDNA sequence, as outlined herein. In a particular embodiment, the Enterobacter species comprises a 16S rDNA sequence that is at least 95% identical to SEQ ID NO: 5. In another embodiment, the Enterobacter species comprises a 16S rDNA sequence that is at least 99% identical to SEQ ID NO: 5. In still another embodiment, the Enterobacter species comprises a 16S rDNA sequence that is identical to SEQ ID NO: 5. In still another embodiment, the Enterobacter species is the isolate FD17.

In another embodiment, the endophytic bacterium is a species of Micrococcus. In a particular embodiment, the Micrococcus species is identified on the basis of its rDNA sequence, as outlined herein. In a particular embodiment, the Micrococcus species comprises a 16S rDNA sequence that is at least 95% identical to SEQ ID NO: 6. In another embodiment, the Micrococcus species comprises a 16S rDNA sequence that is at least 99% identical to SEQ ID NO: 6. In still another embodiment, the Micrococcus species comprises a 16S rDNA sequence that is identical to SEQ ID NO: 6. In still another embodiment, the Micrococcus species is the isolate S2.

In another embodiment, the endophytic bacterium is a species of Bacillus. In a particular embodiment, the Bacillus species is identified on the basis of its rDNA sequence, as outlined herein. In a particular embodiment, the Bacillus species comprises a 16S rDNA sequence that is at least 95% identical to SEQ ID NO: 7. In another embodiment, the Bacillus species comprises a 16S rDNA sequence that is at least 99% identical to SEQ ID NO: 7. In still another embodiment, the Bacillus species comprises a 16S rDNA sequence that is identical to SEQ ID NO: 7. In still another embodiment, the Bacillus species is the isolate S4.

In another embodiment, the endophytic bacterium is a species of Pantoea. In a particular embodiment, the Pantoea species is identified on the basis of its rDNA sequence, as outlined herein. In a particular embodiment, the Pantoea species comprises a 16S rDNA sequence that is at least 95% identical to SEQ ID NO: 8. In another embodiment, the Pantoea species comprises a 16S rDNA sequence that is at least 99% identical to SEQ ID NO: 8. In still another embodiment, the Pantoea species comprises a 16S rDNA sequence that is identical to SEQ ID NO: 8. In still another embodiment, the Pantoea species is the isolate S6.

In another embodiment, the endophytic bacterium is a species of Acinetobacter. In a particular embodiment, the Acinetobacter species is identified on the basis of its rDNA sequence, as outlined herein. In a particular embodiment, the Acinetobacter species comprises a 16S rDNA sequence that is at least 95% identical to SEQ ID NO: 9. In another embodiment, the Acinetobacter species comprises a 16S rDNA sequence that is at least 99% identical to SEQ ID NO: 9. In still another embodiment, the Acinetobacter species comprises a 16S rDNA sequence that is identical to SEQ ID NO: 9. In still another embodiment, the Acinetobacter species is the isolate S9.

In another embodiment, the endophytic bacterium is a species of Paenibacillus. In a particular embodiment, the Paenibacillus species is identified on the basis of its rDNA sequence, as outlined herein. In a particular embodiment, the Paenibacillus species comprises a 16S rDNA sequence that is at least 95% identical to SEQ ID NO: 10. In another embodiment, the Paenibacillus species comprises a 16S rDNA sequence that is at least 99% identical to SEQ ID NO: 10. In still another embodiment, the Paenibacillus species comprises a 16S rDNA sequence that is identical to SEQ ID NO: 10. In still another embodiment, the Paenibacillus species is the isolate S10.

In some cases, the endophytic microbe can be modified. For example, the endophytic microbe can be genetically modified by introduction of a transgene which stably integrates into the bacterial genome. In another embodiment, the endophytic microbe can be modified to harbor a plasmid or episome containing a transgene. In still another embodiment, the microbe can be modified by repeated passaging under selective conditions.

The microbe can be modified to exhibit altered characteristics. In one embodiment, the endophytic microbe is modified to exhibit increased compatibility with chemicals commonly used in agriculture. Agricultural plants are often treated with a vast array of agrichemicals, including fungicides, biocides (anti-bacterial agents), herbicides, insecticides, nematicides, rodenticides, fertilizers, and other agents. Many such agents can affect the ability of an endophytic bacterium to grow, divide, and/or otherwise confer beneficial traits to the plant.

In some cases, it can be important for the microbe to be compatible with agrichemicals, particularly those with fungicidal or antibacterial properties, in order to persist in the plant although, as mentioned earlier, there are many such fungicidal or antibacterial agents that do not penetrate the plant, at least at a concentration sufficient to interfere with the microbe. Therefore, where a systemic fungicide or antibacterial agent is used in the plant, compatibility of the microbe to be inoculated with such agents will be an important criterion.

In one embodiment, spontaneous isolates of microbes which are compatible with agrichemicals can be used to inoculate the plants according to the methods described herein. For example, fungal microbes which are compatible with agriculturally employed fungicides can be isolated by plating a culture of the microbes on a petri dish containing an effective concentration of the fungicide, and isolating colonies of the microbe that are compatible with the fungicide. In another embodiment, a microbe that is compatible with a fungicide is used for the methods described herein. In still another embodiment, a microbe that is compatible with an antibacterial compound is used for the methods described herein. Fungicide compatible microbes can also be isolated by selection on liquid medium. The culture of microbes can be plated on petri dishes without any forms of mutagenesis; alternatively, the microbes can be mutagenized using any means known in the art. For example, microbial cultures can be exposed to UV light, gamma-irradiation, or chemical mutagens such as ethylmethanesulfonate (EMS) prior to selection on fungicide containing media. Finally, where the mechanism of action of a particular fungicide is known, the target gene can be specifically mutated (either by gene deletion, gene replacement, site-directed mutagenesis, etc.) to generate a microbe that is resilient against that particular fungicide. It is noted that the above-described methods can be used to isolate fungi that are compatible with both fungistatic and fungicidal compounds.

It will also be appreciated by one skilled in the art that a plant may be exposed to multiple types of fungicides or antibacterial compounds, either simultaneously or in succession, for example at different stages of plant growth. Where the target plant is likely to be exposed to multiple fungicidal and/or antibacterial agents, a microbe that is compatible with many or all of these agrichemicals can be used to inoculate the plant. A microbe that is compatible with several fungicidal agents can be isolated, for example, by serial selection. A microbe that is compatible with the first fungicidal agent is isolated as described above (with or without prior mutagenesis). A culture of the resulting microbe can then be selected for the ability to grow on liquid or solid media containing the second antifungal compound (again, with or without prior mutagenesis). Colonies isolated from the second selection are then tested to confirm its compatibility to both antifungal compounds.

Likewise, bacterial microbes that are compatible to biocides (including herbicides such as glyphosate or antibacterial compounds, whether bacteriostatic or bactericidal) that are agriculturally employed can be isolated using methods similar to those described for isolating fungicide compatible microbes. In one embodiment, mutagenesis of the microbial population can be performed prior to selection with an antibacterial agent. In another embodiment, selection is performed on the microbial population without prior mutagenesis. In still another embodiment, serial selection is performed on a microbe: the microbe is first selected for compatibility to a first antibacterial agent. The isolated compatible microbe is then cultured and selected for compatibility to the second antibacterial agent. Any colony thus isolated is tested for compatibility to each, or both antibacterial agents to confirm compatibility with these two agents.

The selection process described above can be repeated to identify isolates of the microbe that are compatible with a multitude of antifungal or antibacterial agents.

Candidate isolates can be tested to ensure that the selection for agrichemical compatibility did not result in loss of a desired microbial bioactivity. Isolates of the microbe that are compatible with commonly employed fungicides can be selected as described above. The resulting compatible microbe can be compared with the parental microbe on plants in its ability to promote germination.

### Plant-Endophyte Combinations

In another aspect, the present invention provides for combinations of endophytes and plants. In one embodiment, disclosed is a seed or seedling of an agricultural plant comprising an exogenous endophytic bacterial population that is disposed on an exterior surface of or within the seed or seedling in an amount effective to colonize the plant, and further comprising a formulation that comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient. In another embodiment, the present invention discloses a seed or seedling of an agricultural plant comprising an endophytic bacterial population that is heterologously disposed on an exterior surface of or within the seed or seedling in an amount effective to colonize the plant, and further comprising a formulation that comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

The endophytic bacterial population consists essentially of an endophytic bacterium described herein. In one embodiment, the endophytic bacterium comprises a 16S rRNA nucleic acid sequence that is at least 95% identical, for example, at least 96%, at least 97%, at least 98% identical to a nucleic acid sequence selected from the group consisting of SEQ ID NOs: 1-10. In another embodiment, the endophytic bacterium comprises a 16S rRNA nucleic acid sequence that is at least 99% identical to a nucleic acid sequence selected from the group consisting of SEQ ID NOs: 1-10. In still another embodiment, the endophytic bacterium comprises a 16S rRNA nucleic acid sequence that is identical to a nucleic acid sequence selected from the group consisting of SEQ ID NOs: 1-10.

In one embodiment according to this aspect, disclosed is a seed of an agricultural plant comprising an exogenous endophytic bacterial population that is disposed on an exterior surface of or within the seed in an amount effective to colonize the plant. The bacterial population is considered exogenous to the seed if that particular seed does not inherently contain the bacterial population. Indeed, several of the endophytic microbes described herein have not been detected, for example, in any of the corn seeds sampled, as determined by highly sensitive methods.

In other cases, the present invention discloses a seed of an agricultural plant comprising an endophytic bacterial population that is heterologously disposed on an exterior surface of or within the seed in an amount effective to colonize the plant. For example, the endophytic bacterial population that is disposed on an exterior surface or within the seed can be an endophytic bacterium that may be associated with the mature plant, but is not found on the surface of or within the seed. Alternatively, the endophytic bacterial population can be found in the surface of, or within the seed, but at a much lower number than is disposed.

In some embodiments, a purified endophytes population is used that includes two or more (e.g., 3, 4, 5, 6, 7, 8, 9, 10, 15, 20, 25 or greater than 25) different endophytes, e.g., obtained from different families of plant or fungus, or different genera of plant or fungus, or from the same genera but different species of plant or fungus.

The different endophytes can be obtained from the same cultivar of agricultural plant (e.g., the same maize, wheat, rice, or barley plant), different cultivars of the same agricultural plant (e.g., two or more cultivars of maize, two or more cultivars of wheat, two or more cultivars of rice, or two or more cultivars of barley), or different species of the same type of agricultural plant (e.g., two or more different species of maize, two or more different species of wheat, two or more different species of rice, or two or more different species of barley). In embodiments in which two or more endophytes are used, each of the endophytes can have different properties or activities, e.g., produce different metabolites, produce different enzymes such as different hydrolytic enzymes, confer different beneficial traits, or colonize different elements of a plant (e.g., leaves, stems, flowers, fruits, seeds, or roots). For example, one endophyte can colonize a first and a second endophyte can colonize a tissue that differs from the first tissue.

Combinations of endophytes can be selected by any one or more of several criteria. In one embodiment, compatible endophytes are selected. As used herein, “compatibility” refers to endophyte populations that do not significantly interfere with the growth, propagation, and/or production of beneficial substances of the other. Incompatible endophyte populations can arise, for example, where one of the populations produces or secrets a compound that is toxic or deleterious to the growth of the other population(s). Incompatibility arising from production of deleterious compounds/agents can be detected using methods known in the art, and as described herein elsewhere. Similarly, the distinct populations can compete for limited resources in a way that makes co-existence difficult.

In another embodiment, combinations are selected on the basis of compounds produced by each population of endophytes. For example, the first population is capable of producing siderophores, and another population is capable of producing anti-fungal compounds. In one embodiment, the first population of endophytes or endophytic components is capable of a function selected from the group consisting of auxin production, nitrogen fixation, production of an antimicrobial compound, siderophore production, mineral phosphate solubilization, cellulase production, chitinase production, xylanase production, and acetoin production. In another embodiment, the second population of endophytes or endophytic component is capable of a function selected from the group consisting of auxin production, nitrogen fixation, production of an antimicrobial compound, siderophore production, mineral phosphate solubilization, cellulase production, chitinase production, xylanase production, and acetoin production. In certain combinations, one of the endophytes is capable of using arabinose as a carbon source. In still another embodiment, the first and second populations are capable of at least one different function.

In still another embodiment, the combinations of endophytes are selected for their distinct localization in the plant after colonization. For example, the first population of endophytes or endophytic components can colonize, and in some cases preferentially colonize, the root tissue, while a second population can be selected on the basis of its preferential colonization of the aerial parts of the agricultural plant. Therefore, in one embodiment, the first population is capable of colonizing one or more of the tissues selected from the group consisting of a root, shoot, leaf, flower, and seed. In another embodiment, the second population is capable of colonizing one or more tissues selected from the group consisting of root, shoot, leaf, flower, and seed. In still another embodiment, the first and second populations are capable of colonizing a different tissue within the agricultural plant.

In still another embodiment, combinations of endophytes are selected for their ability to confer one or more distinct fitness traits on the inoculated agricultural plant, either individually or in synergistic association with other endophytes. Alternatively, two or more endophytes induce the colonization of a third endophyte. For example, the first population of endophytes or endophytic components is selected on the basis that it confers significant increase in biomass, while the second population promotes increased drought tolerance on the inoculated agricultural plant. Therefore, in one embodiment, the first population is capable of conferring at least one trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof. In another embodiment, the second population is capable of conferring a trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, and enhanced soil water retention. In still another embodiment, each of the first and second population is capable of conferring a different trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, and enhanced soil water retention.

The combinations of endophytes can also be selected based on combinations of the above criteria. For example, the first population of endophytes can be selected on the basis of the compound it produces (e.g., its ability to fix nitrogen, thus providing a potential nitrogen source to the plant), while the second population can be selected on the basis of its ability to confer increased resistance of the plant to a pathogen (e.g., a fungal pathogen).

In some aspects of the present invention, it is contemplated that combinations of endophytes can provide an increased benefit to the host plant, as compared to that conferred by a single endophyte, by virtue of additive effects. For example, one endophyte strain that induces a benefit in the host plant may induce such benefit equally well in a plant that is also colonized with a different endophyte strain that also induces the same benefit in the host plant. The host plant thus exhibits the same total benefit from the plurality of different endophyte strains as the additive benefit to individual plants colonized with each individual endophyte of the plurality. In one example, a plant is colonized with two different endophyte strains: one provides a 1× increase in biomass when associated with the plant, and the other provides a 2× increase in biomass when associated with a different plant. When both endophyte strains are associated with the same plant, that plant would experience a 3× (additive of 1×+2× single effects) increase in auxin biomass. Additive effects are a surprising aspect of the present invention, as non-compatibility of endophytes may result in a cancelation of the beneficial effects of both endophytes.

In some aspects of the present invention, it is contemplated that a combination of endophytes can provide an increased benefit to the host plant, as compared to that conferred by a single endophyte, by virtue of synergistic effects. For example, one endophyte strain that induces a benefit in the host plant may induce such benefit beyond additive effects in a plant that is also colonized with a different endophyte strain that also induces that benefit in the host plant. The host plant thus exhibits the greater total benefit from the plurality of different endophyte strains than would be expected from the additive benefit of individual plants colonized with each individual endophyte of the plurality. In one example, a plant is colonized with two different endophyte strains: one provides a 1× increase in biomass when associated with a plant, and the other provides a 2× increase in biomass when associated with a different plant. When both endophyte strains are associated with the same plant, that plant would experience a 5× (greater than an additive of 1×+2× single effects) increase in biomass. Synergistic effects are a surprising aspect of the present invention.

As shown in the Examples section below, the endophytic bacterial populations described herein are capable of colonizing the host plant. In certain cases, the endophytic bacterial population can be applied to the plant, for example the plant seed, or by foliar application, and successful colonization can be confirmed by detecting the presence of the bacterial population within the plant. For example, after applying the bacteria to the seeds, high titers of the bacteria can be detected in the roots and shoots of the plants that germinate from the seeds. In addition, significant quantities of the bacteria can be detected in the rhizosphere of the plants. Therefore, in one embodiment, the endophytic microbe population is disposed in an amount effective to colonize the plant. Colonization of the plant can be detected, for example, by detecting the presence of the endophytic microbe inside the plant. This can be accomplished by measuring the viability of the microbe after surface sterilization of the seed or the plant: endophytic colonization results in an internal localization of the microbe, rendering it resistant to conditions of surface sterilization. The presence and quantity of the microbe can also be established using other means known in the art, for example, immunofluorescence microscopy using microbe specific antibodies, or fluorescence in situ hybridization (see, for example, Amann et al. (2001) Current Opinion in Biotechnology 12:231-236, incorporated herein by reference in its entirety). Alternatively, specific nucleic acid probes recognizing conserved sequences from the endophytic bacterium can be employed to amplify a region, for example by quantitative PCR, and correlated to CFUs by means of a standard curve.

In another embodiment, the endophytic microbe is disposed in an amount effective to be detectable in the mature agricultural plant. In one embodiment, the endophytic microbe is disposed in an amount effective to be detectable in an amount of at least about 100 CFU, at least about 200 CFU, at least about 300 CFU, at least about 500 CFU, at least about 1,000 CFU, at least about 3,000 CFU, at least about 10,000 CFU, at least about 30,000 CFU, at least about 100,000 CFU or more in the mature agricultural plant.

In some cases, the endophytic microbe is capable of colonizing particular tissue types of the plant. In one embodiment, the endophytic microbe is disposed on the seed or seedling in an amount effective to be detectable within a target tissue of the mature agricultural plant selected from a fruit, a seed, a leaf, or a root, or portion thereof. For example, the endophytic microbe can be detected in an amount of at least about 100 CFU, at least about 200 CFU, at least about 300 CFU, at least about 500 CFU, at least about 1,000 CFU, at least about 3,000 CFU, at least about 10,000 CFU, at least about 30,000 CFU, at least about 100,000 CFU or more, in the target tissue of the mature agricultural plant.

In some cases, the microbes disposed on the seed or seedling can be detected in the rhizosphere. This may be due to successful colonization by the endophytic microbe, where certain quantities of the microbe is shed from the root, thereby colonizing the rhizosphere. In some cases, the rhizosphere-localized microbe can secrete compounds (such as siderophores or organic acids) which assist with nutrient acquisition by the plant. Therefore, in another embodiment, the endophytic microbe is disposed on the surface of the seed in an amount effective to detectably colonize the soil environment surrounding the mature agricultural plant when compared with a reference agricultural plant. For example, the microbe can be detected in an amount of at least 100 CFU/g DW, for example, at least 200 CFU/g DW, at least 500 CFU/g DW, at least 1,000 CFU/g DW, at least 3,000 CFU/g DW, at least 10,000 CFU/g DW, at least 30,000 CFU/g DW, at least 100,000 CFU/g DW, at least 300,000 CFU/g DW, or more, in the rhizosphere.

The endophytic bacterial populations described herein are also capable of providing many fitness benefits to the host plant. As shown in the Examples section, endophyte-inoculated plants display increased seed germination, increased vigor, increased biomass (e.g., increased root or shoot biomass), increased photochemical efficiency. Therefore, in one embodiment, the endophytic bacterial population is disposed on the surface or within a tissue of the seed or seedling in an amount effective to increase the biomass of the plant, or a part or tissue of the plant grown from the seed or seedling. The increased biomass is useful in the production of commodity products derived from the plant. Such commodity products include an animal feed, a fish fodder, a cereal product, a processed human-food product, a sugar or an alcohol. Such products may be a fermentation product or a fermentable product, one such exemplary product is a biofuel. The increase in biomass can occur in a part of the plant (e.g., the root tissue, shoots, leaves, etc.), or can be an increase in overall biomass. Increased biomass production, such an increase meaning at least about 10%, 20%, 30%, 40%, 50%, 60%, 70%, 80%, 90%, 100%, or greater than 100% when compared with a reference agricultural plant. Such increase in overall biomass can be under relatively stress-free conditions. In other cases, the increase in biomass can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress. In one particular embodiment, the endophytic bacterial population is disposed in an amount effective to increase root biomass by at least 1%, at least 2%, at least 3%, at least 4%, at least 5%, at least 6%, at least 7%, at least 8%, at least 9%, or at least 10%, for example, at least 20%, at least 30%, at least 40%, at least 50%, at least 60%, at least 75%, at least 100%, or more, when compared with a reference agricultural plant.

In another embodiment, the endophytic bacterial population is disposed on the surface or within a tissue of the seed or seedling in an amount effective to increase the rate of seed germination when compared with a reference agricultural plant. For example, the increase in seed germination can be at least 1%, at least 2%, at least 3%, at least 4%, at least 5%, at least 6%, at least 7%, at least 8%, at least 9%, or at least 10%, for example, at least 20%, at least 30%, at least 40%, at least 50%, at least 60%, at least 75%, at least 100%, or more, when compared with a reference agricultural plant.

In other cases, the endophytic microbe is disposed on the seed or seedling in an amount effective to increase the average biomass or yield of the fruit or cob from the resulting plant by at least 1%, at least 2%, at least 3%, at least 4%, at least 5%, at least 6%, at least 7%, at least 8%, at least 9%, or at least 10%, for example, at least 15%, at least 20%, at least 30%, at least 40%, at least 50%, at least 75%, at least 100% or more, when compared with a reference agricultural plant.

As highlighted in the Examples section, plants inoculated with the endophytic bacterial population also show an increase in overall plant height. Therefore, in one embodiment, the present invention provides for a seed comprising an endophytic bacterial population which is disposed on the surface or within a tissue of the seed or seedling in an amount effective to increase the height of the plant. For example, the endophytic bacterial population is disposed in an amount effective to result in an increase in height of the agricultural plant such that is at least 1%, at least 2%, at least 3%, at least 4%, at least 5%, at least 6%, at least 7%, at least 8%, at least 9%, or at least 10% greater, for example, at least 20% greater, at least 30% greater, at least 40% greater, at least 50% greater, at least 60% greater, at least 70% greater, at least 80% greater, at least 90% greater, at least 100% greater, at least 125% greater, at least 150% greater or more, when compared with a reference agricultural plant, the plant. Such increase in height can be under relatively stress-free conditions. In other cases, the increase in height can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress.

The host plants inoculated with the endophytic bacterial population also show dramatic improvements in their ability to utilize water more efficiently. Water use efficiency is a parameter often correlated with drought tolerance. Water use efficiency (WUE) is a parameter often correlated with drought tolerance, and is the CO2 assimilation rate per water transpired by the plant. An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems. An increased water use efficiency of the plant relates in some cases to an increased fruit/kernel size or number.

Therefore, in one embodiment, the plants described herein exhibit an increased water use efficiency when compared with a reference agricultural plant grown under the same conditions. For example, the plants grown from the seeds comprising the endophytic bacterial population can have at least 1% higher WUE, for example, at least 1%, at least 2%, at least 3%, at least 4%, at least 5%, at least 6%, at least 7%, at least 8%, at least 9%, or at least 10% higher, at least 20% higher, at least 30% higher, at least 40% higher, at least 50% higher, at least 60% higher, at least 70% higher, at least 80% higher, at least 90% higher, at least 100% higher WUE than a reference agricultural plant grown under the same conditions. Such an increase in WUE can occur under conditions without water deficit, or under conditions of water deficit, for example, when the soil water content is less than or equal to 60% of water saturated soil, for example, less than or equal to 50%, less than or equal to 40%, less than or equal to 30%, less than or equal to 20%, less than or equal to 10% of water saturated soil on a weight basis.

In a related embodiment, the plant comprising the endophytic bacterial endophyte can have at least 1%, at least 2%, at least 3%, at least 4%, at least 5%, at least 6%, at least 7%, at least 8%, at least 9%, or at least 10% higher relative water content (RWC), for example, at least least 20% higher, at least 30% higher, at least 40% higher, at least 50% higher, at least 60% higher, at least 70% higher, at least 80% higher, at least 90% higher, at least 100% higher RWC than a reference agricultural plant grown under the same conditions.

The endophytes described herein may also confer to the plant an increased ability to grow in nutrient limiting conditions, for example by solubilizing or otherwise making available to the plants macronutrients or micronutrients that are complexed, insoluble, or otherwise in an unavailable form. In one embodiment, a plant is inoculated with an endophyte that confers increased ability to liberate and/or otherwise provide to the plant with nutrients selected from the group consisting of phosphate, nitrogen, potassium, iron, manganese, calcium, molybdenum, vitamins, or other micronutrients. Such a plant can exhibit increased growth in soil containing limiting amounts of such nutrients when compared with reference agricultural plant. Differences between the endophyte-associated plant and reference agricultural plant can be measured by comparing the biomass, or other physical parameters described above, of the two plant types grown under limiting conditions. Therefore, in one embodiment, the plant containing the endophyte able to confer increased tolerance to nutrient limiting conditions exhibits a difference in a physiological parameter that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions of nutrient stress. In another embodiment, the plant containing the endophyte is able to grown under nutrient stress conditions while exhibiting no difference in the physiological parameter compared to a plant that is grown without nutrient stress. In some embodiments, such a plant will exhibit no difference in the physiological parameter when grown with 2-5% less nitrogen than average cultivation practices on normal agricultural land, for example, at least 5-10% less nitrogen, at least 10-15% less nitrogen, at least 15-20% less nitrogen, at least 20-25% less nitrogen, at least 25-30% less nitrogen, at least 30-35% less nitrogen, at least 35-40% less nitrogen, at least 40-45% less nitrogen, at least 45-50% less nitrogen, at least 50-55% less nitrogen, at least 55-60% less nitrogen, at least 60-65% less nitrogen, at least 65-70% less nitrogen, at least 70-75% less nitrogen, at least 80-85% less nitrogen, at least 85-90% less nitrogen, at least 90-95% less nitrogen, or less, when compared with crop plants grown under normal conditions during an average growing season. In some embodiments, the microbe capable of providing nitrogen-stress tolerance to a plant is diazotrophic. In other embodiments, the microbe capable of providing nitrogen-stress tolerance to a plant is non-diazotrophic.

Many of the microbes described herein are capable of producing the plant hormone auxin indole acetic acid (IAA) when grown in culture. Auxin may play a key role in altering the physiology of the plant, including the extent of root growth. Therefore, in another embodiment, the endophytic bacterial population is disposed on the surface or within a tissue of the seed or seedling in an amount effective to detectably induce production of auxin in the agricultural plant. For example, the increase in auxin production can be at least 1%, at least 2%, at least 3%, at least 4%, at least 5%, at least 6%, at least 7%, at least 8%, at least 9%, or at least 10%, for example, at least 20%, at least 30%, at least 40%, at least 50%, at least 60%, at least 75%, at least 100%, or more, when compared with a reference agricultural plant. In one embodiment, the increased auxin production can be detected in a tissue type selected from the group consisting of the root, shoot, leaves, and flowers.

In another embodiment, the endophytic bacterial population of the present invention can cause a detectable modulation in the amount of a metabolite in the plant or part of the plant. Such modulation can be detected, for example, by measuring the levels of a given metabolite and comparing with the levels of the metabolite in a reference agricultural plant grown under the same conditions.

Plants useful for the Present Invention

The methods and compositions according to the present invention can be deployed for any seed plant species. Monocotyledonous as well as dicotyledonous plant species are particularly suitable. The methods and compositions are preferably used with plants that are important or interesting for agriculture, horticulture, for the production of biomass used in producing liquid fuel molecules and other chemicals, and/or forestry.

Thus, the invention has use over a broad range of plants, preferably higher plants pertaining to the classes of Angiospermae and Gymnospermae. Plants of the subclasses of the Dicotylodenae and the Monocotyledonae are particularly suitable. Dicotyledonous plants belong to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

The methods and compositions of the present invention are preferably used in plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-wheat X rye), Bamboo, Carthamus tinctorius (saffiower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (ryegrass).

The methods described herein can also be used with genetically modified plants, for example, to yield additional trait benefits to a plant. For example, a genetically modified plant which is, by means of the transgene, optimized with respect to a certain trait, can be further augmented with additional trait benefits conferred by the newly introduced microbe. Therefore, in one embodiment, a genetically modified plant is contacted with a microbe.

### Formulations/Seed Coating Compositions

In some embodiments, the present invention contemplates seeds comprising a endophytic bacterial population, and further comprising a formulation. The formulation useful for these embodiments generally comprise at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

In some cases, the endophytic bacterial population is mixed with an agriculturally compatible carrier. The carrier can be a solid carrier or liquid carrier. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes the endophytic bacterial population of the present invention (see, for example, U.S. Pat. No. 7,485,451, which is incorporated herein by reference in its entirety). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or plant growth medium. Other agricultural carriers that may be used include fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the formulation can comprise a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some cases, it is advantageous for the formulation to contain agents such as a fungicide, an antibacterial agent, an herbicide, a nematicide, an insecticide, a plant growth regulator, a rodenticide, and a nutrient. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In the liquid form, for example, solutions or suspensions, the endophytic bacterial populations of the present invention can be mixed or suspended in aqueous solutions. Suitable liquid diluents or carriers include aqueous solutions, petroleum distillates, or other liquid carriers.

Solid compositions can be prepared by dispersing the endophytic bacterial populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In one particular embodiment, the formulation is ideally suited for coating of the endophytic microbial population onto seeds. The endophytic bacterial populations described in the present invention are capable of conferring many fitness benefits to the host plants. The ability to confer such benefits by coating the bacterial populations on the surface of seeds has many potential advantages, particularly when used in a commercial (agricultural) scale.

The endophytic bacterial populations herein can be combined with one or more of the agents described above to yield a formulation suitable for combining with an agricultural seed or seedling. The bacterial population can be obtained from growth in culture, for example, using a synthetic growth medium. In addition, the microbe can be cultured on solid media, for example on petri dishes, scraped off and suspended into the preparation. Microbes at different growth phases can be used. For example, microbes at lag phase, early-log phase, mid-log phase, late-log phase, stationary phase, early death phase, or death phase can be used.

The formulations comprising the endophytic bacterial population of the present invention typically contains between about 0.1 to 95% by weight, for example, between about 1% and 90%, between about 3% and 75%, between about 5% and 60%, between about 10% and 50% in wet weight of the bacterial population of the present invention. It is preferred that the formulation contains at least about 10³ per ml of formulation, for example, at least about 10⁴, at least about 10⁵, at least about 10⁶, at least 10⁷ CFU, at least 10⁸ CFU per ml of formulation.

As described above, in certain embodiments, the present invention contemplates the use of endophytic bacteria that are heterologously disposed on the plant, for example, the seed. In certain cases, the agricultural plant may contain bacteria that are substantially similar to, or even genetically indistinguishable from, the bacteria that are being applied to the plant. It is noted that, in many cases, the bacteria that are being applied is substantially different from the bacteria already present in several significant ways. First, the bacteria that are being applied to the agricultural plant have been adapted to culture, or adapted to be able to grow on growth media in isolation from the plant. Second, in many cases, the bacteria that are being applied are derived from a clonal origin, rather than from a heterologous origin and, as such, can be distinguished from the bacteria that are already present in the agricultural plant by the clonal similarity. For example, where a microbe that has been inoculated by a plant is also present in the plant (for example, in a different tissue or portion of the plant), or where the introduced microbe is sufficiently similar to a microbe that is present in some of the plants (or portion of the plant, including seeds), it is still possible to distinguish between the inoculated microbe and the native microbe by distinguishing between the two microbe types on the basis of their epigenetic status (e.g., the bacteria that are applied, as well as their progeny, would be expected to have a much more uniform and similar pattern of cytosine methylation of its genome, with respect to the extent and/or location of methylation).

### Population of Seeds

In another aspect, the invention provides for a substantially uniform population of seeds comprising a plurality of seeds comprising the endophytic bacterial population, as described herein above. Substantial uniformity can be determined in many ways. In some cases, at least 1%, at least 2%, at least 3%, at least 4%, at least 5%, at least 6%, at least 7%, at least 8%, at least 9%, or at least 10%, for example, at least 20%, at least 30%, at least 40%, at least 50%, at least 60%, at least 70%, at least 75%, at least 80%, at least 90%, at least 95% or more of the seeds in the population, contains the endophytic bacterial population in an amount effective to colonize the plant disposed on the surface of the seeds. In other cases, at least 1%, at least 2%, at least 3%, at least 4%, at least 5%, at least 6%, at least 7%, at least 8%, at least 9%, or at least 10%, for example, at least 20%, at least 30%, at least 40%, at least 50%, at least 60%, at least 70%, at least 75%, at least 80%, at least 90%, at least 95% or more of the seeds in the population, contains at least 100 CFU on its surface, for example, at least 200 CFU, at least 300 CFU, at least 1,000 CFU, at least 3,000 CFU, at least 10,000 CFU, at least 30,000 CFU, at least 100,000 CFU, at least 300,000 CFU, or at least 1,000,000 CFU per seed or more.

In a particular embodiment, the population of seeds is packaged in a bag or container suitable for commercial sale. Such a bag contains a unit weight or count of the seeds comprising the endophytic bacterial population as described herein, and further comprises a label. In one embodiment, the bag or container contains at least 1,000 seeds, for example, at least 5,000 seeds, at least 10,000 seeds, at least 20,000 seeds, at least 30,000 seeds, at least 50,000 seeds, at least 70,000 seeds, at least 80,000 seeds, at least 90,000 seeds or more. In another embodiment, the bag or container can comprise a discrete weight of seeds, for example, at least 1 lb, at least 2 lbs, at least 5 lbs, at least 10 lbs, at least 30 lbs, at least 50 lbs, at least 70 lbs or more. The bag or container comprises a label describing the seeds and/or said endophytic bacterial population. The label can contain additional information, for example, the information selected from the group consisting of: net weight, lot number, geographic origin of the seeds, test date, germination rate, inert matter content, and the amount of noxious weeds, if any. Suitable containers or packages include those traditionally used in plant seed commercialization. The invention also contemplates other containers with more sophisticated storage capabilities (e.g., with microbiologically tight wrappings or with gas- or water-proof containments).

In some cases, a sub-population of seeds comprising the endophytic bacterial population is further selected on the basis of increased uniformity, for example, on the basis of uniformity of microbial population. For example, individual seeds of pools collected from individual cobs, individual plants, individual plots (representing plants inoculated on the same day) or individual fields can be tested for uniformity of microbial density, and only those pools meeting specifications (e.g., at least 80% of tested seeds have minimum density, as determined by quantitative methods described elsewhere) are combined to provide the agricultural seed sub-population.

The methods described herein can also comprise a validating step. The validating step can entail, for example, growing some seeds collected from the inoculated plants into mature agricultural plants, and testing those individual plants for uniformity. Such validating step can be performed on individual seeds collected from cobs, individual plants, individual plots (representing plants inoculated on the same day) or individual fields, and tested as described above to identify pools meeting the required specifications.

### Population of Plants/Agricultural Fields

A major focus of crop improvement efforts has been to select varieties with traits that give, in addition to the highest return, the greatest homogeneity and uniformity. While inbreeding can yield plants with substantial genetic identity, heterogeneity with respect to plant height, flowering time, and time to seed, remain impediments to obtaining a homogeneous field of plants. The inevitable plant-to-plant variability are caused by a multitude of factors, including uneven environmental conditions and management practices. Another possible source of variability can, in some cases, be due to the heterogeneity of the microbial population inhabit the plants. By providing endophytic bacterial populations onto seeds and seedlings, the resulting plants generated by germinating the seeds and seedlings have a more consistent microbial composition, and thus are expected to yield a more uniform population of plants.

Therefore, in another aspect, the invention provides a substantially uniform population of plants. The population comprises at least 100 plants, for example, at least 300 plants, at least 1,000 plants, at least 3,000 plants, at least 10,000 plants, at least 30,000 plants, at least 100,000 plants or more. The plants are grown from the seeds comprising the endophytic bacterial population as described herein. The increased uniformity of the plants can be measured in a number of different ways.

In one embodiment, there is an increased uniformity with respect to the microbes within the plant population. For example, in one embodiment, a substantial portion of the population of plants, for example at least 1%, at least 2%, at least 3%, at least 4%, at least 5%, at least 6%, at least 7%, at least 8%, at least 9%, or at least 10%, at least 20%, at least 30%, at least 40%, at least 50%, at least 60%, at least 70%, at least 75%, at least 80%, at least 90%, at least 95% or more of the seeds or plants in a population, contains a threshold number of the endophytic bacterial population. The threshold number can be at least 100 CFU, for example at least 300 CFU, at least 1,000 CFU, at least 3,000 CFU, at least 10,000 CFU, at least 30,000 CFU, at least 100,000 CFU or more, in the plant or a part of the plant. Alternatively, in a substantial portion of the population of plants, for example, in at least 1%, at least 2%, at least 3%, at least 4%, at least 5%, at least 6%, at least 7%, at least 8%, at least 9%, or at least 10%, at least 20%, at least 30%, at least 40%, at least 50%, at least 60%, at least 70%, at least 75%, at least 80%, at least 90%, at least 95% or more of the plants in the population, the endophytic bacterial population that is provided to the seed or seedling represents at least 1%, at least 2%, at least 3%, at least 4%, at least 5%, at least 6%, at least 7%, at least 8%, at least 9%, or at least 10%, least 20%, at least 30%, at least 40%, at least 50%, at least 60%, at least 70%, at least 80%, at least 90%, at least 95%, at least 99%, or 100% of the total microbe population in the plant/seed.

In another embodiment, there is an increased uniformity with respect to a physiological parameter of the plants within the population. In some cases, there can be an increased uniformity in the height of the plants when compared with a population of reference agricultural plants grown under the same conditions. For example, there can be a reduction in the standard deviation in the height of the plants in the population of at least 1%, at least 2%, at least 3%, at least 4%, at least 5%, at least 6%, at least 7%, at least 8%, at least 9%, or at least 10%, at least 15%, at least 20%, at least 30%, at least 40%, at least 50%, at least 60% or more, when compared with a population of reference agricultural plants grown under the same conditions. In other cases, there can be a reduction in the standard deviation in the flowering time of the plants in the population of at least 1%, at least 2%, at least 3%, at least 4%, at least 5%, at least 6%, at least 7%, at least 8%, at least 9%, or at least 10%, at least 15%, at least 20%, at least 30%, at least 40%, at least 50%, at least 60% or more, when compared with a population of reference agricultural plants grown under the same conditions.

### Commodity Plant Product

The present invention provides a commodity plant product, as well as methods for producing a commodity plant product, that is derived from a plant of the present invention. As used herein, a “commodity plant product” refers to any composition or product that is comprised of material derived from a plant, seed, plant cell, or plant part of the present invention. Commodity plant products may be sold to consumers and can be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, paper, tea, coffee, silage, crushed of whole grain, and any other food for human or animal consumption; and biomasses and fuel products; and raw material in industry. Industrial uses of oils derived from the agricultural plants described herein include ingredients for paints, plastics, fibers, detergents, cosmetics, lubricants, and biodiesel fuel. Soybean oil may be split, inter-esterified, sulfurized, epoxidized, polymerized, ethoxylated, or cleaved. Designing and producing soybean oil derivatives with improved functionality and improved oliochemistry is a rapidly growing field. The typical mixture of triglycerides is usually split and separated into pure fatty acids, which are then combined with petroleum-derived alcohols or acids, nitrogen, sulfonates, chlorine, or with fatty alcohols derived from fats and oils to produce the desired type of oil or fat. Commodity plant products also include industrial compounds, such as a wide variety of resins used in the formulation of adhesives, films, plastics, paints, coatings and foams.

In some cases, commodity plant products derived from the plants, or using the methods of the present invention can be identified readily. In some cases, for example, the presence of viable endophytic microbes can be detected using the methods described herein elsewhere. In other cases, particularly where there are no viable endophytic microbes, the commodity plant product may still contain at least a detectable amount of the specific and unique DNA corresponding to the microbes described herein. Any standard method of detection for polynucleotide molecules may be used, including methods of detection disclosed herein.

Throughout the specification, the word “comprise,” or variations such as “comprises” or “comprising,” will be understood to imply the inclusion of a stated integer or group of integers but not the exclusion of any other integer or group of integers.

Although the present invention has been described in detail with reference to examples below, it is understood that various modifications can be made without departing from the spirit of the invention. For instance, while the particular examples below may illustrate the methods and embodiments described herein using a specific plant, the principles in these examples may be applied to any agricultural crop. Therefore, it will be appreciated that the scope of this invention is encompassed by the embodiments of the inventions recited herein and the specification rather than the specific examples that are exemplified below. All cited patents and publications referred to in this application are herein incorporated by reference in their entirety.

### EXAMPLES

### Example 1: Phenotypic and Physiological Characterization of Microbes

Bacterial strains from overnight cultures in tryptic soy broth were streaked on tryptic soy agar (TSA) plates and incubated at 30° C. After 24 h, the color and shape of colonies were noted. Cell motility and colony shape were observed under light microscope (Nikon, Japan). The pH limits for bacterial growth was determined by adjusting the pH of the growth media to values between 5 and 12 in triplicates. Bacterial growth on different salt concentrations was tested in TSA medium containing 1-6% NaCl. Furthermore, the ability of the microbes to grow in methanol/ethanol as sole carbon source was analyzed by replacing the glucose with either methanol or ethanol.

Aggregate formation of bacterial strains can positively affect their dispersal and survival in the plant environment and adsorption to plant roots. The extent of aggregation formation was measured in six replicates following the method of Madi and Henis (1989) Plant Soil 115:89-98 (incorporated herein by reference) with some modifications. Aliquots of liquid culture containing aggregates were transferred to glass tubes and allowed to stand for 30 min. Aggregates settled down to the bottom of each tubes, and the suspension was mostly composed free of cells. The turbidity of each suspension was measured at 540 nm (ODs) with a microplate reader (Synergy 5; BioTek Instrument Inc., Winooski, USA). Cultures were then dispersed with a tissue homogenizer for 1 min and the total turbidity (OD) was measured.

The percentage of aggregation was estimated as follows:

$$\% aggregation = ( ODt - ODs ) \times 100 / ODt$$

Motility assays (swimming, swarming and twitching) were performed following the methods of Rashid and Kornberg (2000). Swim plates (LB media contained 0.3% agarose) were inoculated in triplicates with bacteria from an overnight culture on TSA agar plates grown at 30° C. with a sterile toothpick. For swarming, plates (NB media contained 0.5% agar and glucose) were inoculated with a sterile toothpick. Twitch plates (LB broth containing 1% Difco granular agar) were stab inoculated with a sharp toothpick to the bottom of petri dish from an overnight grown culture in TSA agar plates.

Biofilm formation was analyzed using overnight grown bacterial culture in 96 well microtiter plates by staining with 1% crystal violet (CV) for 45 min. To quantify the amount of biofilm, CV was destained with 200 μl of 100% ethanol. The absorbance of 150 μl of the destained CV, which was transferred into a new microtiter plate was measured at 595 nm (modified from Djordjevic et al. 2002, Appl Environ Microbiol 68:2950-2958, incorporated herein by reference). The phenotypic characters of the strains are shown in Table 1.

TABLE 1
Phenotypic characteristics of the strains:

| | | | | | | |
|---|---|---|---|---|---|---|
| | Agrobacterium | Pantoea | Sphinogobium | Pseudomonas | Enterobacter | Micrococus |
| Characteristics | sp. (FA13) | sp. (FF34) | sp. (FC42) | sp. (FB12) | sp. (FD17) | sp. S2 |

Phenotypic and physiological characterization

| | | | | | | |
|---|---|---|---|---|---|---|
| Colony | Gray | Yellow | Yellow | Gray | Creamy | Creamy |
| color | | | | | white | |
| Colony | Round | Round | Round | Round | Round | Round |
| morphology | | | | | | |
| Gram | n.d. | n.d. | n.d. | n.d. | n.d. | + |
| reaction | | | | | | |

Bacterial growth conditions*
Temperature

| | | | | | | |
|---|---|---|---|---|---|---|
| 4° C. | n.d. | n.d. | n.d. | n.d. | n.d. | − |
| 42° C. | n.d. | n.d. | n.d. | n.d. | n.d. | − |

NaCl

| | | | | | | |
|---|---|---|---|---|---|---|
| 2% | + | + | + | + | + | + |
| 6% | − | + | − | − | + | + |

pH

| | | | | | | |
|---|---|---|---|---|---|---|
| 5 | + | + | + | + | + | + |
| 12 | + | − | − | + | + | + |

Motility/chemotaxist‡

| | | | | | | |
|---|---|---|---|---|---|---|
| Swimming | + | + | − | ++ | +++ | − |
| Swarming | − | − | − | − | + | − |
| Twitching | + | + | − | + | + | − |

Biofilm formation

| | | | | | | |
|---|---|---|---|---|---|---|
| OD | 0.92 ± 0.04 | 0.59 ± 0.02 | 0.95 ± 0.08 | 0.57 ± 0.00 | 0.95 ± 0.04 | 0.92 ± 0.04 |
| (600 nm) | | | | | | |
| Biofilm | 0.23 ± 0.02 | 0.22 ± 0.03 | 0.08 ± 0.01 | 0.08 ± 0.04 | 0.83 ± 0.06 | 0.23 ± 0.02 |
| (595 nm) | | | | | | |
| Aggregate | 35.91 ± 2.57 | 26.07 ± 0.88 | 32.61 ± 2.13 | 36.38 ± 1.48 | 40.22 ± 1.99 | 35.91 ± 2.57 |
| stability | | | | | | |
| (%) | | | | | | |

| | Bacillus | Pantoea | Acinetobacter | Paenibacillus |
|---|---|---|---|---|
| Characteristics | sp. S4 | sp. S6 | sp. S9 | sp. S10 |
| Phenotypic and physiological characterization | | | | |
| Colony | Off- | Yellow | White | Creamy |
| color | white | | | white |
| Colony | Round | Round | Round | Round |
| morphology | | | | |
| Gram | + | − | − | − |
| reaction | | | | |
| Bacterial growth conditions* | | | | |
| Temperature | | | | |
| 4° C. | + | + | + | + |
| 42° C. | − | − | − | − |
| | NaCl | | | |
| 2% | + | + | + | + |
| 6% | + | + | − | + |
| pH | | | | |
| 5 | + | + | + | + |
| 12 | − | + | − | + |
| Motility/chemotaxist‡ | | | | |
| Swimming | − | + | − | ++ |
| Swarming | − | ++ | − | + |
| Twitching | + | + | − | + |
| Biofilm formation | | | | |
| OD | 0.59 ± 0.02 | 0.95 ± 0.08 | 0.57 ± 0.08 | 0.95 ± 0.04 |
| (600 nm) | | | | |
| Biofilm | 0.22 ± 0.03 | 0.08 ± 0.01 | 0.08 ± 0.04 | 0.83 ± 0.06 |
| (595 nm) | | | | |
| Aggregate | 26.07 ± 0.88 | 32.61 ± 2.13 | 36.38 ± 1.48 | 40.22 ± 1.99 |
| stability | | | | |
| (%) | | | | |

### Biochemical Characterization

Biochemical tests such as oxidase, catalase, gelatin hydrolysis and casein hydrolysis of the selected strains were performed. Oxidase and catalase activities were tested with 1% (w/v) tetramethyl-p-phenylene diamine and 3% (v/v) hydrogen peroxide solution, respectively. Gelatin and casein hydrolysis was performed by streaking bacterial strains onto a TSA plates from the stock culture. After incubation, trichloroacetic acid (TCA) was applied to the plates and made observation immediately for a period of at least 4 min (Medina and Baresi 2007, J Microbiol Methods 69:391-393, incorporated herein by reference). A summary of the biochemical characteristics of the strains is shown below in Table 2:

TABLE 2
Biochemical Characterization of Endophytic Bacteria
Biochemical characterization*

| | Agrobacterium | Pantoea | Sphinogobium | Pseudomonas | Enterobacter |
|---|---|---|---|---|---|
| | sp. (FA13) | sp. (FF34) | sp. (FC42) | sp. (FB12) | sp. (FD17) |
| Catalase | + | + | + | + | + |
| Oxidase | − | − | − | + | − |
| Casein | − | − | − | + | − |
| Gelatin | − | + | − | + | + |
| Methanol | + | − | − | + | − |
| Ethanol | + | − | − | + | − |
| | Micrococus | Bacillus | Pantoea | Acinetobacter | Paenibacillus |
| | sp. S2 | sp. S4 | sp. S6 | sp. S9 | sp. S10 |
| Catalase | + | + | + | + | + |
| Oxidase | − | + | − | − | + |
| Casein | + | + | − | − | − |
| Gelatin | + | − | + | − | − |
| Methanol | + | − | − | + | + |
| Ethanol | + | − | − | + | + |

### Quantification of Auxin Production

Auxin production by bacterial isolates both in the presence and absence of L-tryptophan (L-TRP) was determined colormnetrically and expressed as IAA equivalent (Sarwar et al. 1992, Plant Soil 147:207-215, incorporated herein by reference). Two days old bacterial cells grown (28° C. at 180 rpm) in tryptic soy broth supplemented with 1% L-TRP solution were harvested by centrifugation (10, 000 g for 10 min). Three mL of the supernatants were mixed with 2 mL Salkowski's reagent (12 g L⁻¹ FeCl₃ in 429 ml L⁻¹ H₂SO₄). The mixture was incubated at room temperature for 30 min for colour development and absorbance at 535 nm was measured using spectrophotometer. Auxin concentration produced by bacterial isolates was determined using standard curves for IAA prepared from serial dilutions of 10-100 μg mL⁻¹.

TABLE 3
Production of Indole Acetic Acid by Endophytic Bacteria

| | Agrobacterium | Pantoea | Sphinogobium | Pseudomonas | Enterobacter |
|---|---|---|---|---|---|
| Characteristics | sp. (FA13) | sp. (FF34) | sp. (FC42) | sp. (FB12) | sp. (FD17) |
| without L-TRP | 1.74 ± 0.18 | 10.33 ± 0.35 | 4.89 ± 0.78 | 1.63 ± 0.65 | 7.54 ± 1.02 |
| with L-TRP | 16.13 ± 1.05 | 95.34 ± 2.14 | 38.41 ± 1.78 | 7.26 ± 1.05 | 12.30 ± 0.98 |

As shown in Table 3 above, strains FA13, FF34, FC42, FB12 and FD17 were all shown to produce auxin (ranging from 1.63 to 10.33 μg ml-1 in the absence of L-tryptophan), and the level of auxin production was greatly enhanced by the presence of L-tryptophan in the growth medium (at least 7.26 μg ml-1).

### Assays for Phosphorus Solubilization and Siderophore Production

Bacterial strains were evaluated for their ability to solubilize phosphates (organic/inorganic P). Aliquots (10 μL) of overnight bacterial growth culture in tryptic soy broth were spot inoculated onto NBRI-PBP (Mehta and Nautiyal 2001) and calcium/sodium phytate agar medium (Rosado et al. 1998). Solubilization of organic/inorganic phosphates was detected by the formation of a clear zone around the bacterial growth spot. Phosphate solubilization activity was also determined by development of clear zone around bacterial growth on Pikovskaya agar medium (Pikovskaya 1948, Mikrobiologiya 17:362-370, incorporated herein by reference). Bacterial isolates were assayed for siderophores production on the Chrome azurol S (CAS) agar medium described by Schwyn and Neilands (1987), Curr Microbiol 43:57-58 (incorporated herein by reference) as positive for siderophore production.

### Assays for Exopolysaccharide, NH₃ and HCN Production

For exopolysaccharide (EPS) activity (qualitative), strains were grown on Weaver mineral media enriched with glucose and production of EPS was assessed visually (modified from Weaver et al. 1975, Arch Microbiol 105:207-216, incorporated herein by reference). The EPS production was monitored as floc formation (fluffy material) on the plates after 48 h of incubation at 28±2° C. Strains were tested for the production of ammonia (NH₃) in peptone water as described by Cappuccino and Sherman (1992), Biochemical activities of microorganisms. In: Microbiology, A Laboratory Manual. The Benjamin/Cummings Publishing Co. California, USA, pp 125-178, incorporated herein by reference. The bacterial isolates were screened for the production of hydrogen cyanide (HCN) by inoculating King's B agar plates amended with 4.4 g L⁻¹ glycine (Lorck 1948, Physiol Plant 1:142-146, incorporated herein by reference). Filter paper (Whatman no. 1) saturated with picrate solution (2% Na₂CO₃ in 0.5% picric acid) was placed in the lid of a petri plate inoculated with bacterial isolates. The plates were incubated at 28±2° C. for 5 days. HCN production was assessed by the colour change of yellow filter paper to reddish brown.

Assays for Poly-Hydroxybutyrate (PHB) and n-Acyl-Homoserine Lactone (AHL) Production

The bacterial isolates were tested for PHB production (qualitative) following the viable colony staining methods using Nile red and Sudan black B (Juan et al. 1998 Appl Environ Microbiol 64:4600-4602; Spiekermann et al. 1999, Arch Microbiol 171:73-80, each of which is incorporated by reference). The LB plates with overnight bacterial growth were flooded with 0.02% Sudan black B for 30 min and then washed with ethanol (96%) to remove excess strains from the colonies. The dark blue coloured colonies were taken as positive for PHB production. Similarly, LB plates amended with Nile red (0.5 μL mL⁻¹) were exposed to UV light (312 nm) after appropriate bacterial growth to detect PHB production. Colonies of PHA-accumulating strains showed fluoresce under ultraviolet light. The bacterial strains were tested for AHL production following the method modified from Cha et al. (1998), Mol Plant-Microbe Interact 11:1119-1129 (incorporated herein by reference). The LB plates containing 40 μg ml⁻¹ X-Gal were plated with reporter strains (A. tumefaciens NTL4.pZLR4). The LB plates were spot inoculated with 10 μL of bacterial culture and incubated at 28±2° C. for 24 h. Production of AHL activity is indicated by a diffuse blue zone surrounding the test spot of culture. Agrobacterium tumefaciens NTL1 (pTiC58ΔaccR) was used as positive control and plate without reporter strain was considered as negative control.

TABLE 4
Various Biochemical Properties of Endophytic Bacteria

| | | | | | |
|---|---|---|---|---|---|
| | Agrobacterium | Pantoea | Sphinogobium | Pseudomonas | Enterobacter |
| Characteristics^{†} | sp. (FA13) | sp. (FF34) | sp. (FC42) | sp. (FB12) | sp. (FD17) |

P-solubilization
(inorganic/organic P)

| Ca₃(PO₄)₂ | − | ++ | − | + | +++ |
|---|---|---|---|---|---|
| CaHPO₄ | − | ++ | − | + | +++ |
| Ca-phytate | − | ++ | − | ++ | +++ |
| Na-phytate | − | ++ | − | ++ | +++ |
| Exopolysaccharide | ++ | − | + | − | + |
| N₂-fixation | + | + | − | − | + |
| HCN production | − | − | − | + | − |
| NH₃ production | + | + | + | + | + |
| Siderophore | +++ | + | + | ++ | +++ |
| production | | | | | |
| AHL | − | − | − | + | − |
| PHB | − | + | − | + | + |
| | Micrococus | Bacillus | Pantoea | Acinetobacter | Paenibacillus |
| Characteristics^{†} | sp. S2 | sp. S4 | sp. S6 | sp. S9 | sp. S10 |

P-solubilization
(inorganic/organic P)

| | | | | | |
|---|---|---|---|---|---|
| Ca₃(PO₄)₂ | − | − | + | − | + |
| CaHPO₄ | − | − | + | − | + |
| Ca-phytate | − | − | + | − | + |
| Na-phytate | − | − | + | − | + |
| Exopolysaccharide | − | − | | − | + |
| N₂-fixation | − | − | + | − | − |
| HCN production | − | − | − | − | − |
| NH₃ production | + | + | + | + | + |
| Siderophore | n.d. | − | n.d. | − | + |
| production | | | | | |
| AHL | − | − | + | − | + |
| PHB | + | − | + | − | − |

As shown above, the bacteria described herein exhibit varying degrees of phosphate utilization. For example, strains FF34, FB12, FD17, S6, and S10 were capable of hydrolyzing Ca₃(PO₄)₂, CaHPO₄, Ca-phytate and Na-phytate. These strains, therefore, may be effective for increasing phosphate availability for host plants under conditions of limiting concentrations of soluble phosphate in the soil.

Siderophores are small, high-affinity iron chelating compounds secreted by microorganisms such as bacteria, fungi and grasses, siderophores. They bind to the available form of iron Fe³⁺ in the rhizosphere, thus making it unavailable to the phytopathogens and protecting the plant health (Ahmad et al. 2008, Microbiol Res 163:173-181, incorporated herein by reference). Siderophores are known for mobilizing Fe and making it available to the plant. Several of the strains, including FA13, FF34, FC42, FB12, FD17 and S10 were found to produce significant levels of siderophore when tested in agar medium containing Chrom azurol S (CAS). Therefore, in one embodiment, the strains described above are effective in increasing iron availability to the host plant.

The ability of bacterial strains to utilize or metabolize different nitrogen sources was evaluated. Interestingly, four of the strains tested (FA13, FF34, FD17, and S6) were capable of growing in nitrogen-free medium, demonstrating their ability to fix nitrogen. Therefore, in one embodiment, these strains can be provided in an amount effective to increase nitrogen utilization in a host plant.

Bacterial survival and colonization in the plant environment are necessary for plant growth and yield. Recently, Zúñiga and colleagues (2013), Mol Plant-Microbe Interact 26:546-553 (incorporated herein by reference) described that the cell-to-cell communication (QS) system mediated by AHL is implicated in rhizosphere competence and colonization of Arabidopsis thaliana by B. phytofirmans PsJN. Motility, aggregate stability, and biofilm formation are important traits for root surface colonization (Danhorn and Fuqua 2007, Annu Rev Microbiol 61:401-422, incorporated herein by reference). Three strains (FB12, S6 and S10) were found to produce AHL. It should be noted, however, that the bacteria described here may have other communication systems. Aggregation and biofilm formation were common traits in all tested strains. In the case of motility, six strains (FA13, FF34, FB12, FD17, S6 and S10) were positive for swimming, while FD17, S6 and S10 also showed swarming. Therefore, in one embodiment, the seeds are provided with an amount of these strains in an amount effective to produce detectable levels of AHL. In another embodiment, seeds of an agricultural plant are provided with an amount of the bacterial endophyte population effective to form biofilms.

Bacteria were tested for production of exopolysaccharide (EPS) and poly-hydroxybutyrate (PHB). Bacterial EPS and PHB have been shown to provide protection from such environmental insults as desiccation, predation, and the effects of antibiotics (Gasser et al. 2009, FEMS Microbiol Ecol 70:142-150; Staudt et al. 2012, Arch Microbiol 194:197-206, each of which is incorporated by reference). They can also contribute to bacterial aggregation, surface attachment, and plant-microbe symbiosis (Laus et al. 2005, Mol Plant-Microbe Interact 18:533-538, incorporated herein by reference). Five strains (FF34, FB12, FD17, S2 and S6) showed PHB production, while FA13, FC42, FD17 and S10 were found to produce EPS. Therefore, in another embodiment, seeds of an agricultural plant are provided with an amount of the bacterial endophyte population effective to improve desiccation tolerance in the host plant.

Volatile compounds such as ammonia and HCN produced by a number of rhizobacteria were reported to play an important role in biocontrol (Brimecombe et al. 2001, In: Pinton R, Varanini Z, Nannipieri P (Eds.) The Rhizosphere, Marcel Dekker, New York, pp 95-140, incorporated herein by reference). Production of ammonia was commonly detected in all selected isolates. In contrast, only Pseudomonas sp. strain FB12 was able to produce HCN. Among the strains tested, only FB12 was able to produce HCN.

### Enzyme Hydrolyzing Activities

Bacterial hydrolyzing activities due to amylase, cellulase, chitinase, hemolytic, lipase, pectinase, protease and xylanase were screened on diagnostic plates after incubation at 28° C. Amylase activity was determined on agar plates following the protocol Männistö and Häggblom (2006), Syst Appl Microbiol 29:229-243, incorporated herein by reference. Formation of opaque halo around colonies was used as an indication of lipase activity. Cellulase and xylanase activities were assayed on plates containing (per liter) 5 g of carboxymethyl cellulose or birch wood xylan, 1 g of peptone and 1 g of yeast extract. After 10 days of incubation, the plates were flooded with gram's iodine staining and washing with 1M NaCl to visualize the halo zone around the bacterial growth (modified from Teather and Wood 1982, Appl Environ Microbiol 43:777-780, incorporated herein by reference). Chitinase activity of the isolates was determined as zones of clearing around colonies following the method of Chernin et al. (1998) J Bacteriol 180:4435-4441 (incorporated herein by rereference). Hemolytic activity was determined by streaking bacterial isolates onto Cloumbia 5% sheep blood agar plates. Protease activity was determined using 1% skimmed milk agar plates, while lipase activity was determined on peptone agar medium. Formation of halo zone around colonies was used as indication of activity (Smibert and Krieg 1994, In: Gerhardt P, Murray R, Wood W, Krieg N (Eds) Methods for General and Molecular Bacteriology, ASM Press, Washington, DC, pp 615-640, incorporated herein by reference). Pectinase activity was determined on nutrient agar supplemented with 5 g I¹ pectin. After 1 week of incubation, plates were flooded with 2% hexadecyl trimethyl ammonium bromide solution for 30 min. The plates were washed with 1M NaCl to visualize the halo zone around the bacterial growth (Mateos et al. 1992, Appl Environ Microbiol 58:1816-1822, incorporated herein by reference).

TABLE 5
Enzyme Activities from Endophytic Bacteria

| | | | | | |
|---|---|---|---|---|---|
| | Agrobacterium | Pantoea | Sphinogobium | Pseudomonas | Enterobacter |
| Characteristics | sp. (FA13) | sp. (FF34) | sp. (FC42) | sp. (FB12) | sp. (FD17) |

Enzyme hydrolyzing activity^{‡}

| Amylase | − | − | − | − | − |
|---|---|---|---|---|---|
| Cellulase | + | − | + | + | ++ |
| Chitinase | − | − | − | + | + |
| Hemolytic | + | + | − | + | + |
| Lipase | ++ | + | + | +++ | ++ |
| Pectinase | − | + | − | + | + |
| Phosphatase | − | ++ | − | ++ | +++ |
| Protease | − | − | − | − | − |
| Xylanase | + | − | +++ | + | ++ |
| | Micrococus | Bacillus | Pantoea | Acinetobacter | Paenibacillus |
| Characteristics | sp. S2 | sp. S4 | sp. S6 | sp. S9 | sp. S10 |

Enzyme hydrolyzing activity^{‡}

| | | | | | |
|---|---|---|---|---|---|
| Amylase | − | − | − | − | + |
| Cellulase | + | + | − | − | + |
| Chitinase | − | + | − | − | − |
| Hemolytic | n.d. | n.d. | n.d. | n.d. | n.d. |
| Lipase | − | + | + | + | + |
| Pectinase | − | − | + | − | + |
| Phosphatase | − | − | + | − | + |
| Protease | + | + | − | − | − |
| Xylanase | + | + | + | − | + |

All strains showed lipase activity, while only S10 produced amylase activity. S2 and S4 produced significant protease activity. Pectinase and phosphatase activity was observed with strains FF34, FB12, FD17, S6 and S10. All strains were positive for cellulase and/or xylanase except strains FF34 and S9. Chitinase was produced by FB12, FD17 and S4 strains, while all strains tested except for FC42 showed hemolytic activity.

### Antagonistic Activities Against Plant Pathogenic Bacteria, Fungi and Oomycetes

The antagonistic activities of bacterial isolates were screened against plant pathogenic bacteria (Agrobacterium tumefaciens, Pseudomonas syringae, Streptococcus pneumoniae), fungi (Fusarium caulimons, Fusarium graminarium, Fusarium oxysporum, Fusarium solani, Rhizoctonia solani, Thielaviopsis basicola) and oomycetes (Phytophthora infestans, Phytophthora citricola, Phytophthora cominarum). For antibacterial assays, the bacterial isolates and pathogen were cultivated in tryptic soy broth at 30° C. for 24 h. The bacterial isolates were spot-inoculated (10 μL aliquots) on TSA plates pre-seeded with 100 μL tested pathogen. The plates were incubated at 28° C. for 48 h and clear zones of inhibition were recorded.

Antagonistic activity of the bacterial isolates against fungi and oomycetes was tasted by the dual culture technique on potato dextrose agar (PDA) and yeast malt agar (YMA) media (Dennis and Webster 1971, Trans Brit Mycol Soc 57:25-39, incorporated herein by reference). A small disk (5 mm) of target fungus/oomycetes was placed in the center of petri dishes of both media. Aliquots of 10 μL of overnight bacterial cultures grown in tryptic soy broth were spotted 2 cm away from the center. Plates were incubated for 14 days at 24° C. and zones of inhibition were scored.

TABLE 6
Antimicrobial Activity by Endophytic Bacteria

| | | | | | |
|---|---|---|---|---|---|
| | Agrobacterium | Pantoea | Sphinogobium | Pseudomonas | Enterobacter |
| Characteristics | sp. (FA13) | sp. (FF34) | sp. (FC42) | sp. (FB12) | sp. (FD17) |

Anti-bacterial activity

| | | | | | |
|---|---|---|---|---|---|
| A. tumefaciens | − | − | − | ++ | + |
| E. coli | n.d. | n.d. | n.d. | n.d. | n.d. |
| P. syringae | − | − | − | +++ | + |
| S. aureus | − | − | − | + | − |

Anti-fungal activity

| | | | | | |
|---|---|---|---|---|---|
| F. caulimons | ++ | + | + | ++ | +++ |
| F. graminarium | + | + | + | + | ++ |
| F. oxysporum | + | ++ | + | ++ | ++ |
| F. solani | ++ | + | ++ | ++ | +++ |
| R. solani | + | + | + | ++ | ++ |
| T. basicola | + | + | + | ++ | + |

Anti-oomycete activity

| P. infestans | + | + | + | ++ | ++ |
|---|---|---|---|---|---|
| P. citricola | + | + | + | ++ | +++ |
| P. cominarum | + | + | + | ++ | ++ |
| | Micrococus | Bacillus | Pantoea | Acinetobacter | Paenibacillus |
| Characteristics | sp. S2 | sp. S4 | sp. S6 | sp. S9 | sp. S10 |

Anti-bacterial activity

| | | | | | |
|---|---|---|---|---|---|
| A. tumefaciens | − | + | − | − | + |
| E. coli | + | + | − | − | + |
| P. syringae | − | + | − | − | + |
| S. aureus | + | + | + | + | + |

Anti-fungal activity

| | | | | | |
|---|---|---|---|---|---|
| F. caulimons | − | + | + | − | + |
| F. graminarium | − | − | + | + | + |
| F. oxysporum | + | + | + | − | − |
| F. solani | − | + | − | − | + |
| R. solani | + | + | + | + | + |
| T. basicola | − | + | + | − | + |

Anti-oomycete activity

| | | | | | |
|---|---|---|---|---|---|
| P. infestans | − | − | + | − | − |
| P. citricola | − | − | + | + | + |
| P. cominarum | − | + | + | + | + |

### Auxin, Acetoin, and Siderophore Assays for S4, S9 and S10

For the auxin assay, 1 μl of overnight-grown cultures of endophytic bacterial strains were inoculated into 750 μl of R2A broth supplemented with L-TRP (5 mM) in 2-mL 96 well culture plates. The plates were sealed with a breathable membrane and incubated at 23° C. with constant shaking at 200 rpm for 4 days. After 4 days, 100 μL of each culture was transferred to a 96 well plate. 25 μL of Salkowski reagent (1 mL of FeCl3 0.5 M solution to 50 mL of 35% HClO4) was added into each well and the plates were incubated in the dark for 30 minutes before taking picture and measuring 540 nm obsorption using the SpectraMax M5 plate reader (Molecular Devices).

For acetoin measurements, microbial strains were cultured as described above in R2A broth supplemented with 5% glucose. After 4 days, 100 μL of each culture was transferred to a 96 well plate and mixed with 25 μL Barritt's Reagents (See Example 3) and 525 nm absorption was measured.

For siderophore measurements, microbial strains were cultured as described above in R2A broth. After 3 days of incubation at 28° C. without shaking, to each well was added 100 ul of O-CAS preparation without gelling agent [Perez-Miranda et al. (2007), J Microbiol Methods 70: 127-131, incorporated herein by reference]. Again using the cleaned glassware, 1 liter of O-CAS overlay was made by mixing 60.5 mg of Chrome azurol S (CAS), 72.9 mg of hexadecyltrimethyl ammonium bromide (HDTMA), 30.24 g of finely crushed Piperazine-1,4-bis-2-ethanesulfonic acid (PIPES) with 10 ml of 1 mM FeCl3·6H2O in 10 mM HCl solvent. The PIPES had to be finely powdered and mixed gently with stirring (not shaking) to avoid producing bubbles, until a dark blue colour was achieved. 15 minutes after adding the reagent to each well, color change was scored by looking for purple halos (catechol type siderophores) or orange colonies (hydroxamate siderophores) relative to the deep blue of the O-Cas.

The results of the auxin, acetoin and siderophore assays are presented in Table 7.

TABLE 7
Production of auxin, acetoin and siderophore
by endophytic bacteria

| | S4 | S9 | S10 |
|---|---|---|---|
| auxin | ++ | ++ | + |
| acetoin | − | − | +++ |
| siderophore | ++ | ++ | +++ |

### Substrate Use by Endophytic Bacteria S4, S9 and S10

In addition to determining whether the bacterial strains produce auxin, acetoin, and siderophores, the ability of the various strains to grow on various substrates was determined. Liquid cultures of bacteria were first sonicated to achieve homogeneity. 1 mL culture of each strain was harvested by centrifugation for 10 minutes at 4500 RPM and subsequently washed three times with sterile distilled water to remove any traces of residual media. Bacterial samples were resuspended in sterile distilled water to a final OD590 of 0.2. Measurements of absorbance were taken using a SpectraMax M microplate reader (Molecular Devices, Sunnyvale, CA).

Sole carbon substrate assays were done using BIOLOG Phenotype MicroArray (PM) 1 and 2A MicroPlates (Hayward, CA). An aliquot of each bacterial cell culture (2.32 mL) were inoculated into 20 mL sterile IF-0a GN/GP Base inoculating fluid (IF-0), 0.24 mL 100× Dye F obtained from BIOLOG, and brought to a final volume of 24 mL with sterile distilled water. Negative control PM1 and PM2A assays were also made similarly minus bacterial cells to detect abiotic reactions. An aliquot of fungal culture (0.05 mL) of each strain were inoculated into 23.95 mL FF-IF medium obtained from BIOLOG. Bacterial cell suspensions were stirred in order to achieve uniformity. One hundred microliters of the bacterial cell suspension was added per well using a multichannel pipettor to the 96-well BIOLOG PM1 and PM2A MicroPlates that each contained 95 carbon sources and one water-only (negative control) well.

MicroPlates were sealed in paper surgical tape (Dynarex, Orangeburg, NY) to prevent plate edge effects, and incubated stationary at 24° C. in an enclosed container for 70 hours. Absorbance at 590 nm was measured for all MicroPlates at the end of the incubation period to determine carbon substrate utilization for each strain and normalized relative to the negative control (water only) well of each plate (Garland and Mills, 1991; Barua et al., 2010; Siemens et al., 2012; Blumenstein et al., 2015). The bacterial assays were also calibrated against the negative control (no cells) PM1 and PM2A MicroPlates data to correct for any biases introduced by media on the colorimetric analysis (Borglin et al., 2012). Corrected absorbance values that were negative were considered as zero for subsequent analysis (Garland and Mills, 1991; Blumenstein et al., 2015) and a threshold value of 0.1 and above was used to indicate the ability of a particular bacterial strain to use a given carbon substrate (Barua et al., 2010; Blumenstein et al., 2015). Additionally, bacterial MicroPlates were visually examined for the irreversible formation of violet color in wells indicating the reduction of the tetrazolium redox dye to formazan that result from cell respiration (Garland and Mills, 1991).

The results of these assays are shown in Tables 8 (BIOLOG PM1 MicroPlates) and 9 (BIOLOG PM2A MicroPlates).

TABLE 8
Substrate utilization of certain endophytic strains
as determined by BIOLOG PM1 MicroPlates.

| substrate | SYM260 | SYM290 | SYM292 |
|---|---|---|---|
| D-Serine | NO | NO | YES |
| D-Glucose-6-Phosphate | NO | NO | NO |
| L-Asparagine | YES | NO | NO |
| L-glutamine | NO | NO | NO |
| Glycyl-L-Aspartic acid | NO | NO | NO |
| Glycyl-L-Glutamic acid | YES | NO | NO |
| Glycyl-L-Proline | NO | NO | NO |
| L-Arabinose | YES | YES | YES |
| D-Sorbitol | NO | NO | NO |
| D-Galactonic acid-γ-lactone | NO | NO | NO |
| D-Aspartic acid | NO | NO | NO |
| m-Tartaric acid | NO | NO | NO |
| Citric acid | YES | NO | YES |
| Tricarballylic acid | NO | NO | NO |
| p-Hydroxy Phenyl acetic acid | NO | NO | NO |
| N-Acetyl-D-Glucosamine | NO | YES | YES |
| Glycerol | YES | YES | YES |
| D-L-Malic acid | YES | YES | YES |
| D-Glucosaminic acid | NO | NO | NO |
| D-Glucose-1-Phosphate | NO | NO | NO |
| m-Inositol | YES | NO | YES |
| L-Serine | YES | NO | NO |
| m-Hydroxy Phenyl Acetic acid | NO | NO | NO |
| D-Saccharic acid | YES | NO | YES |
| L-Fucose | NO | YES | NO |
| D-Ribose | NO | YES | YES |
| 1,2-Propanediol | YES | YES | NO |
| D-Fructose-6-Phosphate | NO | NO | NO |
| D-Threonine | NO | YES | NO |
| L-Threonine | YES | YES | NO |
| Tyramine | NO | NO | YES |
| Succinic acid | NO | NO | NO |
| D-Glucuronic acid | NO | NO | NO |
| Tween 20 | YES | YES | NO |
| Tween 40 | YES | YES | NO |
| Tween 80 | YES | YES | NO |
| Fumaric acid | YES | YES | YES |
| L-Alanine | YES | YES | YES |
| D-Psicose | NO | NO | NO |
| D-Galactose | NO | YES | YES |
| D-Gluconic acid | YES | YES | YES |
| L-Rhamnose | NO | YES | YES |
| a-Keto-Glutaric acid | YES | NO | YES |
| a-Hydroxy Glutaric acid-γ-lactone | YES | NO | NO |
| Bromo succinic acid | YES | NO | YES |
| L-Alanyl-Glycine | YES | YES | YES |
| L-Lyxose | NO | NO | NO |
| L-Aspartic acid | YES | NO | YES |
| D-L-a-Glycerol phosphate | YES | NO | NO |
| D-Fructose | NO | YES | YES |
| a-Keto-Butyric acid | NO | NO | NO |
| a-Hydroxy Butyric acid | YES | YES | NO |
| Propionic acid | YES | YES | YES |
| Acetoacetic acid | YES | YES | YES |
| Glucuronamide | NO | YES | NO |
| L-Proline | YES | NO | YES |
| D-Xylose | YES | YES | YES |
| Acetic acid | YES | YES | YES |
| a-Methyl-D-Galactoside | NO | YES | YES |
| β-Methyl-D-glucoside | YES | YES | YES |
| Mucic acid | YES | NO | YES |
| N-acetyl-β-D-Mannosamine | YES | YES | YES |
| Pyruvic acid | YES | YES | YES |
| D-Alanine | NO | YES | NO |
| L-Lactic acid | YES | NO | YES |
| a-D-Glucose | NO | YES | YES |
| a-D-Lactose | NO | YES | YES |
| Adonitol | NO | NO | NO |
| Glycolic acid | YES | NO | NO |
| Mono Methyl Succinate | YES | YES | YES |
| L-Galactonic-acid-γ-lactone | YES | YES | YES |
| D-Trehalose | NO | YES | YES |
| Formic acid | YES | NO | YES |
| Maltose | YES | YES | YES |
| Lactulose | NO | YES | YES |
| Maltotriose | YES | YES | YES |
| Glyoxylic acid | YES | NO | YES |
| Methyl Pyruvate | YES | YES | YES |
| D-Galacturonic acid | YES | NO | YES |
| D-Mannose | NO | NO | YES |
| D-Mannitol | YES | YES | YES |
| D-Melibiose | NO | YES | YES |
| Sucrose | NO | YES | YES |
| 2-Deoxy adenosine | YES | NO | YES |
| D-Cellobiose | YES | YES | YES |
| D-Malic acid | YES | NO | YES |
| Phenylethyl-amine | NO | NO | NO |
| Dulcitol | NO | YES | NO |
| L-Glutamic acid | NO | NO | NO |
| Thymidine | YES | YES | YES |
| Uridine | YES | YES | YES |
| Adenosine | YES | YES | YES |
| Inosine | NO | NO | YES |
| L-Malic acid | YES | NO | YES |

TABLE 9
Substrate utilization of certain endophytic strains
as determined by BIOLOG PM2A MicroPlates.

| substrate | SYM260 | SYM290 | SYM292 |
|---|---|---|---|
| Negative control | N/A | N/A | N/A |
| N-acetyl-D-Galactosamine | NO | NO | NO |
| Gentiobiose | YES | YES | YES |
| D-Raffinose | YES | YES | YES |
| Capric acid | NO | NO | NO |
| D-lactic acid methyl ester | NO | NO | NO |
| Acetamide | NO | NO | NO |
| L-Ornithine | YES | NO | NO |
| Chondrointin sulfate C | YES | NO | NO |
| N-acetyl-neuraminic acid | NO | NO | NO |
| L-glucose | NO | NO | NO |
| Salicin | YES | YES | YES |
| Caproic acid | YES | NO | YES |
| Malonic acid | YES | NO | NO |
| L-Alaninamide | NO | YES | NO |
| L-Phenylalanine | YES | NO | NO |
| a-Cyclodextrin | NO | YES | YES |
| β-D-allose | NO | NO | YES |
| Lactitol | NO | YES | YES |
| Sedoheptulosan | NO | NO | NO |
| Citraconic acid | NO | NO | NO |
| Melibionic acid | YES | NO | NO |
| N-Acetyl-L-Glutamic acid | YES | NO | YES |
| L-Pyroglutamic acid | YES | NO | YES |
| β-Cyclodextrin | NO | YES | YES |
| Amygdalin | NO | YES | YES |
| D-Melezitose | NO | YES | YES |
| L-Sorbose | NO | NO | NO |
| Citramalic acid | YES | NO | YES |
| Oxalic acid | NO | NO | NO |
| L-Arginine | YES | NO | NO |
| L-Valine | YES | NO | YES |
| α-Cyclodextrin | NO | YES | YES |
| D-arabinose | NO | YES | YES |
| Maltitol | NO | YES | YES |
| Stachyose | YES | YES | YES |
| D-Glucosamine | YES | YES | YES |
| Oxalomalic acid | YES | YES | YES |
| Glycine | NO | NO | NO |
| D,L-Carnitine | NO | NO | NO |
| Dextrin | YES | YES | YES |
| D-arabitol | NO | NO | YES |
| a-Methyl-D-Glucoside | NO | YES | YES |
| D-Tagatose | NO | YES | NO |
| 2-Hydroxy benzoic acid | NO | NO | NO |
| Quinic acid | NO | NO | NO |
| L-Histidine | YES | YES | NO |
| Sec-Butylamine | NO | NO | NO |
| Gelatin | YES | YES | YES |
| L-arabitol | NO | NO | NO |
| β-Methyl-D-Galactoside | NO | YES | YES |
| Turanose | NO | YES | YES |
| 4-Hydroxy benzoic acid | NO | NO | NO |
| D-Ribono-1,4-Lactone | NO | NO | NO |
| L-Homoserine | NO | NO | NO |
| D,L-Octopamine | NO | NO | NO |
| Glycogen | YES | YES | YES |
| Arbutin | NO | YES | YES |
| 3-Methyl Glucose | NO | NO | YES |
| Xylitol | NO | NO | YES |
| β-Hydroxy butyric acid | YES | NO | NO |
| Sebacic acid | YES | NO | NO |
| Hydroxy-L-Proline | YES | NO | YES |
| Putrescine | YES | NO | NO |
| Inulin | YES | YES | YES |
| 2-Deoxy-D-Ribose | NO | NO | YES |
| β-Methyl-D-Glucuronic acid | NO | NO | YES |
| N-Acetyl-D-glucosaminitol | NO | NO | NO |
| γ-Hydroxy butyric acid | YES | NO | NO |
| Sorbic acid | NO | NO | NO |
| L-Isoleucine | YES | NO | YES |
| Dihydroxy acetone | NO | NO | YES |
| Laminarin | NO | YES | YES |
| i-Erythritol | NO | NO | NO |
| a-Methyl-D-Mannoside | NO | NO | NO |
| γ-amino butyric acid | YES | NO | NO |
| a-Keto-valeric acid | YES | NO | NO |
| Succinamic acid | NO | NO | NO |
| L-Leucine | NO | NO | YES |
| 2,3-Butanediol | YES | NO | NO |
| Mannan | NO | NO | NO |
| D-Fucose | NO | NO | NO |
| β-Methyl-D-Xyloside | NO | YES | YES |
| d-amino valeric acid | YES | NO | NO |
| Itaconic acid | YES | NO | YES |
| D-Tartaric acid | NO | NO | NO |
| L-Lysine | YES | NO | NO |
| 2,3-Butanone | NO | NO | NO |
| Pectin | NO | YES | YES |
| 3-0-β-D-Galactopyranosyl-D- | NO | NO | YES |
| arabinose | | | |
| Palatinose | NO | YES | YES |
| Butyric acid | YES | NO | NO |
| 5-Keto-D-Gluconic acid | NO | NO | NO |
| L-Tartaric acid | NO | NO | NO |
| L-Methionine | NO | NO | NO |

### Example 2: Effect of Endophytic Strains on Maize Germination

Inoculants of the selected strains were prepared in 50 mL tryptic soy broth in 100 mL Erlenmeyer flasks and incubated at 28±2° C. for 48 h in the orbital shaking incubator (VWR International, GmbH) at 180 r min⁻¹. The optical density of the broth was adjusted to 0.5 measured at 600 nm using spectrophotometer (Gene Quant Pro, Gemini BV, The Netherlands) to obtain a uniform population of bacteria (10⁸-10⁹ colony-forming units (CFU) mL¹) in the broth at the time of inoculation. More scientifically, harvested bacterial cells could be resuspended in the phosphate buffered saline. The inoculum density adjusts using a spectrophotometer to achieve population density (Pillay and Nowak 1997, Can J Microbiol 43:354-361, incorporated herein by reference).

Maize seeds were surface-sterilized with 70% ethanol (3 min), treated with 5% NaOHCl for 5 min, and followed by washing 3 times with sterile distilled water (1 min each time). The efficacy of surface sterilization was checked by plating seed, and aliquots of the final rinse onto LB plates. Samples were considered to be successfully sterilized when no colonies were observed on the LB plates after inoculation for 3 days at 28° C. Surface-disinfected seeds of different maize cultivars (Helmi, Morignon, Pelicon, Peso and Cesor) were immersed in the bacterial suspensions for 30 min. The bacterized seeds were deposited onto soft water-agar plates (0.8%, w/v agar) and plates were placed in the dark at room temperature (24±2° C.). After 96 hrs the percentage of germinated seeds was scored. Surface-sterilized seeds, but not bacterized (treated in tryptic soy broth), served as the germination control.

Inoculation of maize seeds with endophytic bacteria increased the germination rate of all cultivars by 20-40% compared to the un-inoculated control. Maximum increase was observed by inoculation with strain FD17 (40%) in maize cv. Morignon followed by strains FF34, FA13, FB12 and FC42 (data not shown).

In other experiments, seeds of different cultivars of Maize (Palazzo & die Samba), and Tomato (Red Pear and Gartenfreund) were used to test for promotion of germination. The results, provided below in Table 10, show that virtually all strains show a marked increase in germination rates. For maize, Palazzo seeds inoculated with the strains FA13, FF34, S2, S6, S9 and S10 show greater than 90% germination after four days, as did die Samba seeds inoculated with FF34 and S9 seeds. For tomato, Red Pear seeds inoculated with the strains FB12, FF34, S6 and S10 showed 90% or greater germination rate after 12 days.

TABLE 10
Germination rate of maize and tomato
seeds inoculated with endophytes

| | Maize Germination Rate | | Tomato Germination Rate | |
|---|---|---|---|---|
| | (4 Days) | | (12 days) | |
| | | Maize | Tomato | |
| | Maize | “die | “Red | Tomato |
| Strain | “Palazzo” | Samba” | Pear” | “Gartenfreund” |
| Neg. | 73.3% | 73.3% | 33.3% | 50.0% |
| control | | | | |
| FA13 | 100.0% | 86.7% | 83.3% | 60.0% |
| FB12 | 83.3% | 76.7% | 96.7% | 53.3% |
| FC42 | 86.7% | 86.7% | 76.7% | 80.0% |
| FD17 | 76.7% | 66.7% | 43.3% | 46.7% |
| FF34 | 93.3% | 93.3% | 96.7% | 50.0% |
| S2 | 93.3% | 70.0% | 70.0% | 60.0% |
| S4 | 70.0% | 86.7% | 76.7% | 66.7% |
| S6 | 90.0% | 80.0% | 100.0% | 70.0% |
| S9 | 96.7% | 96.7% | 60.0% | 53.3% |
| S10 | 93.3% | 80.0% | 90.0% | 76.7% |

### Example 3: In Vitro Screening of Efficient Strains on Maize Plants

A growth chamber experiment was conducted on maize to screen the selected strains for their growth promoting activity under gnotobiotic conditions. We used specially designed glass tubes with beaded rim (Duran group, DURAN GmbH, Mainz, Germany) for the experiment. The glass tubes were covered with lid to generate fully axenic conditions (no exposure to any environmental factors). Bacterial inoculant production and seed treatment were done as described above. As control, seeds were treated with sterilized tryptic soy broth. Treated seeds were placed onto water-agar plates for germination. After 5 days, germinated seedlings (3-5 cm long) were transferred in the sterilized glass tubes containing sterilized 20 ml MS (Murashige and Skoog) medium (Duchefa Biochemie, The Netherlands) (4.8 g L⁻¹) and placed at 25±2° C. set at a 16 h light and 8 h dark period, with a light intensity of 350 μmol m⁻² s⁻¹. Data regarding shoot/root length and biomass were recorded after 24 days. Colonization of inoculant strains was scored by re-isolation of endophytes. One g of plant shoot was homogenized with a pestle and mortar in 4 ml of 0.9% (w/v) NaCl solution. The number of cultivable endophytes in maize shoot, expressed in CFU per gram (fresh weight), was determined by spreading serial dilution up to 10⁻⁴ (0.1 mL) of homogenized surface-sterilized plant material onto TSA (DIFCO Laboratories, Detroit, Michigan) agar medium. Four replicates for each treatment were spread on the agar plates and incubated for 5 days at 28° C. Twenty colonies per treatment were randomly selected and their identity with the inoculant strain was confirmed by restriction fragment length polymorphism (RFLP) analysis of the 16S-23S rRNA intergenic spacer (IGS) region (Reiter et al. 2001, Appl Environ Microbiol 68:2261-2268, incorporated herein by reference).

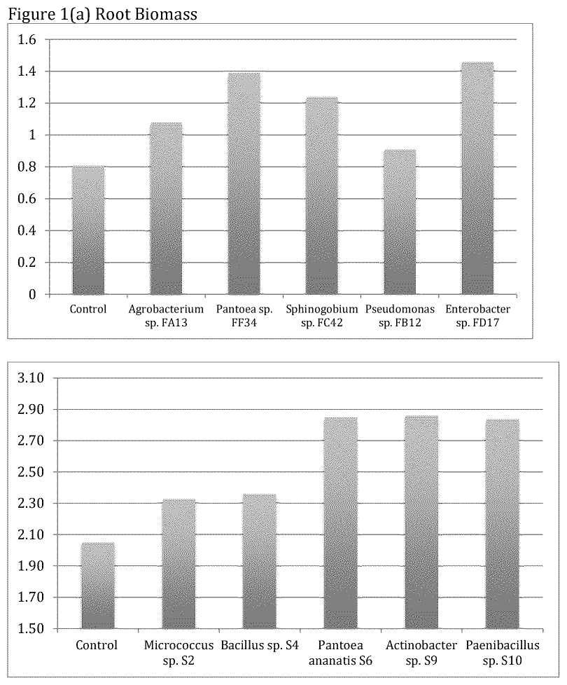

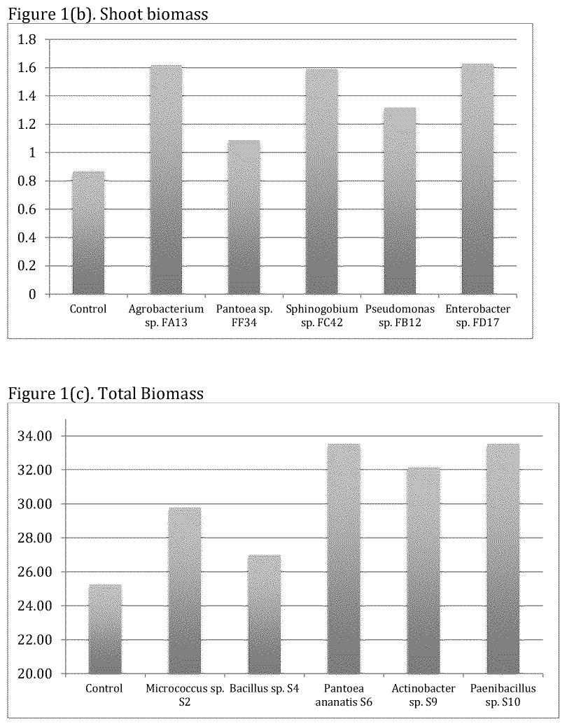

All strains significantly increased the seedling growth compared to the control. As shown in FIGS. 1A-1C, all strains significantly promoted biomass production, with increases in both root, shoot or overall biomass. Though responses were variable, the strains generally increased root and shoot length in all three cultivars of maize tested.

Next, colonization of plants was tested for all bacterial strains. As shown in Table 11, strains FA13, FF34, FC42, FB12 and FD17 successfully colonized corn plants, showing successful colonization of the various strains, as detected in the shoot tissue of various cultivars of maize. The amount of detectable bacteria in the shoot tissue varied, ranging from 1.58×10⁴ in FB12-inoculated Helmi cultivar, to 1.83×10⁷ CFU found in Peso cultivars inoculated with FF34. Therefore, the microbes described herein, when contacted with seeds of plants, are capable of colonizing the plant as detectable, in this case, in the shoot tissue. Furthermore, colonization of Kolea, Mazurka and DaSilvie cultivars of maize by strains S2, S4, S6, S9 and S10 was confirmed by isolating bacterial cells from homogenates of surface sterilized shoot tissue of plants grown from inoculated seeds on tryptic soy agar plates for two days on 28° C. and testing the identity of colonies with IGS region sequencing to confirm the presence of the microbe. S2, S4, S6, S9 and S10 strains were successfully recovered from the tissues of these cultivars (data not shown).

TABLE 11
Colonization of Maize Plants by Endophytic Bacteria

| Strains | Helmi | Peso | Pelicon | Morignon | Cesor |
|---|---|---|---|---|---|
| FA13 | 1.95 × 10⁵ | 1.16 × 10⁷ | 1.2 × 10⁴ | 1.21 × 10⁶ | 3.31 × 10⁶ |
| FF34 | 2.66 × 10⁶ | 1.83 × 10⁷ | 1.21 × 10⁵ | 4.13 × 10⁶ | 9.1 × 10⁶ |
| FC42 | 4.63 × 10⁵ | 1.37 × 10⁶ | 2.00 × 10⁴ | 8.24 × 10⁶ | 1.07 × 10⁵ |
| FB12 | 1.58 × 10⁴ | 1.94 × 10⁷ | 1.12 × 10⁵ | 1.46 × 10⁶ | 9.38 × 10⁵ |
| FD17 | 1.92 × 10⁶ | 2.60 × 10⁷ | 1.44 × 10⁷ | 2.93 × 10⁷ | 1.73 × 10⁶ |

### Stomatal Conductance and Photosynthesis Rates

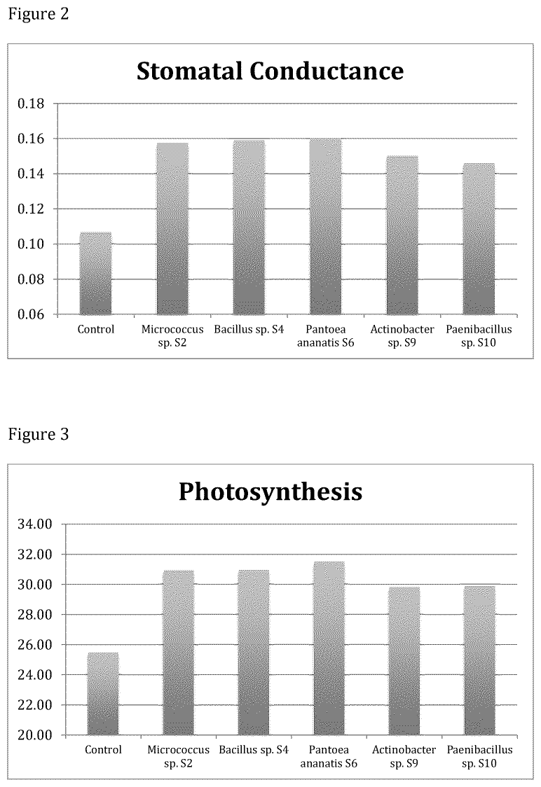

Maize plants inoculated with the strains described herein were tested for photosynthesis and stomatal conductance. As shown in FIG. 2, maize plants inoculated with the strains display an increase in stomatal conductance when compared with uninoculated controls (ranging from a 36% to 49% increase), with S2, S6, S9 strains displaying the highest level of conductance. Therefore, there is an appreciable increase in stomatal conductance conferred by the bacterial of the present invention.

Strain-inoculated maize plants were also tested for photosynthetic rates. As shown in FIG. 3, all strains conferred increased photosynthesis rates when compared with control plants in all three maize cultivars tested (DaSilvie, Mazurka, and Kolea cultivars; average of three cultivars shown), with an increase ranging from 17% over controls (for S9 and S10 strains) to over 23% over controls (S6 strain). Therefore, the endophytic bacterial strains described above confer increased photosynthesis rates on the host plants.

### Example 4: Net-House Experiment

On the basis of the results from tests performed under axenic conditions in Example 3, strain FD17 was selected for further evaluation in a pot trial, in which plants were grown in large containers exposed to natural environmental conditions.

Maize plants were grown in soil collected from agricultural (maize) fields in Fischamend, Lower Austria, Austria. The soil was silty clay loam and had the following characteristics: 12% sand, 61% silt, 27% clay, pH 6.5, 3.3% total carbon, 0.18% total nitrogen, 0.13 mg g⁻¹ available phosphorus, 0.066 mg g⁻¹ extractable potassium.

Surface-disinfected seeds of two maize cultivars (Morignon and Peso) were immersed in bacterial suspension (prepared as described above) for 1 h. For the un-inoculated control, seeds were treated with sterilized tryptic soy broth. Seeds were sown in a plastic tray (wiped with ethanol) and 12 days old seedlings were transferred into containers filled with 45 kg soil (2 plants in each container) and placed in a net-house and exposed to natural environmental conditions.

Weather conditions i.e. precipitation, temperature and relative humidity were recorded by ‘Zentralanstalt fir Meteorologie und Geodynamik’ (ZAMG) during the crop growth period and described in FIGS. 1A-1C. There were three replicates and the pots were arranged in a completely randomized design. Recommended dose of NPK fertilizers (160-100-60 kg ha⁻¹) were applied in each container and tap water was applied to the container for irrigation whenever needed.

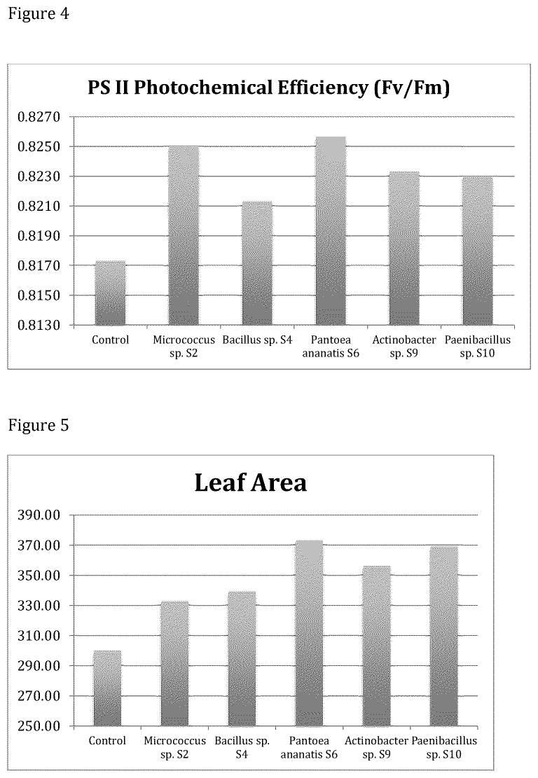

Data of photochemical efficiency of PSII was recorded at flowering stage using handy PEA (Hansatech Instruments Ltd. England) in the mid of July where day time temperature varied from 30-35° C. The PSII efficiency in terms of Fᵥ/Fₘ was calculated from the data. Growth and yield contributing parameters were recorded at maturity. The plants were harvested 140 days after planting. FIG. 4 shows the PS II efficiency of maize plants inoculated with the bacterial endophyte populations described herein.

Maize plants inoculated with the bacterial endophytes S2, S4, S6, S9, S10 and FD17 were tested for increased leaf area. As shown in FIG. 5, and in Table 12, all the tested strains increased the leaf area significantly over the controls.

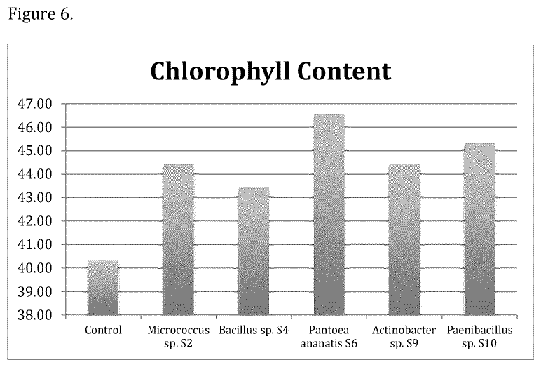

Similarly, maize plants inoculated with the strains showed a dramatic increase in chlorophyll content (FIG. 6) over control plants, with the highest levels found in S6 inoculated plants.

Table 12 below shows the effect of FD17 inoculation on the physiology, growth parameters and yield of two maize cultivars grown in field soil and exposed to natural climatic conditions. Inoculation with strain FD17 led to a significant increase in leaf area of both cultivars (20% and 13%, respectively). Similarly, biomass (leaf dry weight) was increased by 27% and 23% in the cultivars Peso and Morignon, respectively, as compared to the control. In addition, root and plant dry biomass and plant height were significantly enhanced, as was the average cob weight (35% and 42% increase in Peso and Morignon, respectively, as compared to control). The FD17 strain also significantly affected other plant physiological characteristics: for example, there was a significant increase in chlorophyll fluorescence (up to a 9% in the Peso cultivar) and a shortened time before onset of flowering (up to 10 days in cultivar Peso).

TABLE 12
Effect of inoculation with endophytic strain FD17 on
physiology, growth parameters and yield of two maize
cultivars grown in pots in field soil and exposed to
natural climatic conditions (net house experiment)

| | Treatment | | | |
|---|---|---|---|---|
| | Peso | | Morignon | |
| | Un- | Inoculated | Un- | Inoculated |
| Parameters | inoculated | with FD17 | inoculated | with FD17 |
| Fv/Fm | 0.69 | 0.75 | 0.73 | 0.79 |
| Time to onset of | 65.33 | 55 | 70.67 | 66.33 |
| flowering (days) | | | | |
| Plant height (cm) | 192.33 | 208 | 196.69 | 213.68 |
| No. of leaves plant | 12.33 | 14 | 13.17 | 14.67 |
| Leaf area (cm²) | 494.26 | 556.27 | 512.39 | 617.11 |
| Leaf dry weight (g) | 22.21 | 28.16 | 28.09 | 34.56 |
| Plant dry biomass (g) | 114.18 | 153.77 | 160.46 | 223.14 |
| Root dry biomass (g) | 17.26 | 24.34 | 19.73 | 28.28 |
| Cob weight (g) | 115.28 | 155.83 | 123.71 | 176.23 |

Rhizosphere and endophytic colonization of roots, stems and leaves by the inoculant strain were determined by plate counting using TSA plates. Root, stem and leave samples were washed, surface sterilized (as described above) and used for inoculant strain recovery (colonization). For this, samples were crushed in 0.9% (w/v) NaCl solution, shaked with a pulsifier (Microgen Bioproducts Ltd., UK) for 30 see and different dilutions were spread on TSA plates. Bacterial colonies were counted after 4 days of incubation at 28±2° C. The selected colonies were identified and confirmed by IGS region-based RFLP analysis.

The ability of strain FD17 to colonize various tissues of the host plant, as well as the rhizosphere surrounding the plant, was examined. As shown in Table 13 below, seeds of two different maize cultivars inoculated with the FD17 strain resulted in effective, detectable colonization in the root, shoot and leaf interior. Therefore, the seeds were treated with an amount of the endophytic bacterium that is sufficient to colonize the leaf, root, and shoot tissues. Surprisingly, the rhizosphere also had significant levels of detectable FD17. This suggests that the beneficial effects of endophytic bacterial strains such as FD17 could be exerting effects externally to the plant. As described elsewhere, the bacteria described herein are capable of producing compounds which allow increased availability of limiting nutrients such as phosphate and iron. The strains could be present on the surface of the seeds in an amount sufficient to efficiently colonize the plant, but also the surrounding rhizosphere. The presence of significant amounts of detectable bacteria in the rhizosphere raises the interesting possibility that the seeds can be treated with the microbes either on its surface or inside the seed in an amount sufficient to alter the rhizosphere of the plant, thereby altering the soil around the plant, and rendering it more hospitable for the plant.

TABLE 13
Colonization of strain FD17 in rhizosphere root, stem and
leaves of two maize cultivars (wire-house experiment)

| | Plant compartment | | | |
|---|---|---|---|---|
| | Rhizosphere | Root interior | Shoot interior | Leaf interior |
| | (cfu g⁻¹ | (cfu g⁻¹ | (cfu g⁻¹ | (cfu g⁻¹ |
| Maize cv. | dry wt) | dry wt) | dry wt) | dry wt) |
| Peso | 4.07 × 10⁴ | 3.39 × 10⁴ | 1.63 × 10³ | 1.16 × 10² |
| Morignon | 9.85 × 10⁴ | 8.59 × 10⁴ | 3.72 × 10³ | 6.23 × 10² |

### Statistical Analyses

The data of plant growth parameters and colonization were subjected to analyses of variance. The means were compared by Least Significant Difference (LSD) test (p<0.05) to detect statistical significance among treatment (Steel et al. 1997, Principles and procedures of statistics: A biometrical approach. 3rd ed. McGraw-Hill Book Int. Co., Singapore, incorporated herein by reference). All of the statistical analyses were conducted using SPSS software version 19 (IBM SPSS Statistics 19, USA).

### Example 5: Field Trials in Austria

### Methods:

Four varieties of maize were grown at two locations in Austria. Six replicate plots were sown for each treatment and variety combination. Control plots were planted with formulation treated seeds (20 mM phosphate buffer pH 7, 3% sucrose, 1% sodium alginate).

Seeds were sown in a rainfed field in plots arranged in a randomized complete block design. Leaf color was visually assessed at one of the two locations and ranked from 1-3 light green to dark green. Both male and female flowering was visually rated from 0-2 (0=not visible, 1=flower visible, 2=fully developed flower). Corn was hand harvested over 4 m of interior rows. Kernel weight per and kernel moisture per plot were recorded as well as the number of ears harvested per plot. Yield was calculated as kernel weight per plot divided by the number of ears harvested and adjusted for moisture content to a storage moisture of 14% (i.e. dry kernel weight per ear in g).

Results: As shown in Table 14, no treatment related differences were evident for seedling emergence, LAI, color, height and female flower development. A slight increase in male flower development was recorded. No treatment related differences were evident for yield measured as dry kernels per ear in g.

TABLE 14
Rainfed trials in Austria.
Field trial metrics aggregated over both locations and all
varieties. Units: Color (ratings scale 1-3), Flowering
(ratings scale, 0 = not visible, 1 = flower visible, 2 = fully
developed flower), yield (dry kernels per ear in g)

| | | Male | Female | |
|---|---|---|---|---|
| Treatment | Color | Flower | Flower | Yield |
| Formulation | 1.79 | 1.47 | 1.14 | 155.46 |
| control | | | | |
| S2 | 1.95 | 1.54 | 1.12 | 153.83 |
| S6 | 1.79 | 1.52 | 1.06 | 155.17 |
| S9 | 1.95 | 1.61 | 1.04 | 155.49 |
| S10 | 1.87 | 1.52 | 1.14 | 153.29 |
| FD17 | 1.83 | 1.5 | 1.02 | 151.76 |

### Example 6: Field Trials in the US on Maize

### Methods:

Two varieties of maize were grown at one location in the United States in an irrigated trial. Six replicate plots were sown for each treatment and variety combination. Control plots were planted for formulation treated seeds (20 mM phosphate buffer pH 7, 3% sucrose, 1% sodium alginate).

Seeds were sown in an irrigated field in plots of 10 by 40 ft arranged in a randomized complete block design with a JD 7100 cone seeder and box seeder planters respectively. Four rows were planted per plot with a row spacing of 30 inches. Seeding density at was 35,000 seeds per acre. The interior 2 rows were harvested by combine and 10 individual ears were hand harvested from exterior rows. Grain yield per plot, grain moisture, and test weight were assessed. Yield was adjusted for grain moisture content to a storage moisture of 14% (i.e. dry bushels per acre for combine harvest and dry kernels per ear in g for hand harvest).

### Results:

As shown in Table 15, S4, S10, FD17 showed positive impacts on hand harvest yield of up to 10 g per ear. None of the treatments showed a difference when harvested by combine.

TABLE 15
Field trial metrics aggregated over both varieties.
Units: Hand harvest yield (dry kernels per ear in g),
Combine yield (dry bushels per acre).

| | Hand | |
|---|---|---|
| | harvest | Combine |
| Treatment | yield | yield |
| Formulation | 106.58 | 104.66 |
| control | | |
| S2 | 101.40 | 95.79 |
| S4 | 116.04 | 99.29 |
| S6 | 101.68 | 104.58 |
| S9 | 109.16 | 104.30 |
| S10 | 113.34 | 95.28 |
| FD17 | 113.99 | 101.40 |

### Example 7: Field Trials in the US on Spring Wheat

### Methods:

One variety of spring wheat were grown at one location in the United States. Six replicate plots were sown for each treatment and variety combination. Control plots were planted for formulation treated seeds (20 mM phosphate buffer pH 7, 3% sucrose, 1% sodium alginate).

Seeds were sown with a great plains drill in either irrigated or rainfed field in plots of by 40 ft arranged in a randomized complete block design. 7 rows were planted per plot with a row spacing of 17 inches and a seeding density was 60 pounds per acre for rainfed. Wheat was harvested by combine over the entire plot area. Grain yield per plot, grain moisture, and test weight were assessed. Yield was adjusted for grain moisture content to a storage moisture of 13% (i.e. dry bushels per acre).

Results: The combine yield results are shown in Table 16. S6, S10 and S4 showed slight positive trends up to 2 bushels per acre, while S9 showed a statistically significant yield increase of 2 bushels per acre or 4 bushels per acre compared to the formulation controls respectively.

TABLE 16
Units: Combine yield (dry bushels per acre).

| Treatment | Yield |
|---|---|
| Formulation | 40.64 |
| control | |
| S2 | 39.99 |
| S4 | 41.10 |
| S6 | 41.23 |
| S9 | 42.76* |
| S10 | 41.07 |
| FD17 | 38.63 |

### Example 8: Field Trials in Argentina on Maize

### Methods:

Two varieties of maize were grown at one location in Argentina. Ten replicate plots were sown for each treatment and variety combination. Control plots were planted for formulation treated seeds (20 mM phosphate buffer pH 7, 3% sucrose, 1% sodium alginate).

Seeds were sown with a cone planter in a drip irrigated field. The field was located in an extremely arid environment and received irrigation targeted at 80% of evapotranspiration in order to create a managed water stress environment designed to reduce yield by around 20%. Plots were 5×3 m in size and arranged in rectangular blocks in a randomized complete block design. Four rows were planted per plot with a row spacing of 70 cm and an in-row seed spacing of 15 cm. Above and belowground biomass were assessed for 10 plants per plot one month after sowing. The date at which 50% of the plants per plot reached flowering was visually assessed. The interior two rows were harvested by combine. Grain yield per plot, grain moisture and test weight were assessed. Yield was adjusted for grain moisture content to a storage moisture of 14% (i.e. dry bushels per acre).

### Results:

In this trial with moderate water stress, S4 and S9 showed a positive impact on aboveground biomass up to 30 g compared to the formulation control (Table 17). S4 showed a positive increase in belowground biomass compared to the formulation control up to 6 g. S4, S9 and S10 showed a positive increase in yield compared to the formulation controls of up to 19 bushels per acre.

TABLE 17
Managed moderate water stress trial.
Units: Biomass (g), Combine yield (dry bushels per acre).

| | Aboveground | Belowground | |
|---|---|---|---|
| Treatment | biomass | biomass | Yield |
| Formulation | 477.90 | 55.62 | 152.38 |
| control | | | |
| S4 | 503.60 | 61.23 | 163.80 |
| S9 | 486.20 | 56.24 | 171.19* |
| S10 | 466.70 | 57.25 | 163.89 |

1.-20. (canceled)

21. An agricultural formulation comprising a purified bacterial population and an agriculturally acceptable carrier, the bacterial population comprising a first bacterial endophyte capable of at least one of: production of an auxin, nitrogen fixation, production of an antimicrobial, production of a siderophore, mineral phosphate solubilization, production of a cellulase, production of a chitinase, production of a xylanase, utilization of arabinose as a carbon source, and production of acetoin, or a combination of two or more thereof, wherein the first bacterial endophyte comprises a 16S rRNA nucleic acid sequence at least 95% identical to a nucleic acid sequence selected from the group consisting of SEQ ID NOs: 1-10, and wherein the first bacterial endophyte is present in an amount effective to confer a benefit to an agricultural plant seed to which the formulation is applied or to an agricultural plant seedling to which the formulation is applied.

22. The agricultural formulation of claim 21, wherein the purified bacterial population comprises:
(a) two or more species of bacterial endophytes;
(b) two or more families of bacterial endophytes; and/or
(c) a second bacterial endophyte having a 16S nucleic acid sequence less than 95% identical to that of the first bacterial endophyte.

23. The agricultural formulation of claim 21, wherein the formulation is:
(a) a liquid and the bacterial concentration is from about 10\hat{ }3 to about 10\hat{ }11 CFU/ml; or
(b) a gel or powder and the bacterial concentration is from about 10\hat{ }3 to about 10\hat{ }11 CFU/gm.

24. The agricultural formulation of claim 21, wherein:
(a) the benefit is selected from the group consisting of: increased root biomass, increased root length, increased height, increased shoot length, increased leaf number, increased water use efficiency, increased tolerance to low nitrogen stress, increased nitrogen use efficiency, increased overall biomass, increase grain yield, increased photosynthesis rate, increased tolerance to drought, increased heat tolerance, increased salt tolerance, increased resistance to nematode stress, increased resistance to a fungal pathogen, increased resistance to a bacterial pathogen, increased resistance to a viral pathogen, a detectable modulation in the level of a metabolite, and a detectable modulation in the proteome relative to a reference plant, or a combination thereof; and/or
(b) the bacterial endophyte exhibits: production of auxin, production of a siderophore, mineral phosphate solubilization, production of a cellulase, production of a chitinase, production of a xylanase, and production of acetoin, but not increase nitrogen fixation relative to a reference plant.

25. A seed or seedling of an agricultural plant having disposed on an exterior surface of the seed or seedling the formulation of claim 21 comprising an exogenous endophytic bacterial population.

26. The seed or seedling of claim 25, wherein the exogenous endophytic bacterial population within the formulation is disposed in an amount effective:
(a) to be detectable within a target tissue of the mature agricultural plant selected from a fruit, a seed, a leaf, a stem, a shoot, a flower, or a root, or portion thereof,
(b) to be detectable in an amount of at least about 100 CFU of the endophytic bacterial population per gram fresh weight of the target tissue;
(c) to be detectable in an amount of at least about 100 CFU in the mature agricultural plant;
(d) to increase fruit or grain biomass or yield from the resulting plant by at least 5% when compared with a reference agricultural plant;
(e) to increase the biomass of the agricultural plant or portion thereof by at least 5% when compared with a reference agricultural plant;
(f) to increase the rate of seed germination when compared with a reference agricultural plant; and/or
(g) to detectably induce production of auxin in the seed or seedling.

27. The seed or seedling of claim 25, wherein:
(a) the agricultural plant is a monocot or a dicot; or
(b) the seed is a corn seed, and wherein the formulation further comprises at least about 10,000 CFU of the exogenous endophytic bacterial population comprises an endophytic bacterium comprising a 16S rRNA nucleic acid sequence at least 95% identical to a nucleic acid sequence selected from the group consisting of SEQ ID NOs: 1-10 disposed on the exterior surface of the seed, the formulation comprising a microbial stabilizer.

28. A method for preparing a seed or seedling comprising an endophytic bacterial population, said method comprising applying to a seed or seedling a formulation comprising an endophytic bacterial population comprising an endophytic bacterium comprising a 16S rRNA nucleic acid sequence at least 95% identical to a nucleic acid sequence selected from the group consisting of SEQ ID NOs: 1-10.

29. The method of claim 28, wherein the formulation further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

30. The method of claim 28, wherein the endophytic bacterial population is applied:
(a) in an amount effective to be detectable within a target tissue of the mature agricultural plant selected from a fruit, a seed, a leaf, a stem, a shoot, a flower, or a root, or portion thereof,
(b) in an amount effective to be detectable in an amount of at least about 100 CFU of the endophytic bacterial population per gram fresh weight of the target tissue;
(c) is applied in an amount effective to increase water use efficiency of a plant grown from the seed by at least 5% when compared with a reference agricultural plant grown under the same conditions;
(d) is applied in an amount effective to increase photosynthetic rates of a plant grown from the seed by at least 8% when compared with a reference agricultural plant grown under the same conditions;
(e) is applied in an amount effective to increase the biomass of a plant, or plant part, grown from the seed under abiotic-stress free conditions by at least 5% when compared with a reference agricultural plant grown under the same conditions;
(f) population is applied in an amount effective to increase fruit or grain biomass or yield from the resulting plant by at least 5% when compared with a reference agricultural plant that was grown under stress-free conditions;
(g) is applied in an amount effective to increase the rate of seed germination by at least 5% when compared with a reference agricultural plant grown under the same conditions; or
(h) is applied in an amount effective to increase plant height by at least 5% when compared with a reference agricultural plant grown under the same conditions.

31. The method of claim 28, wherein:
(a) the seed is a monocot seed;
(b) the seed is a corn seed; or
(c) the seed is a corn seed, and wherein the formulation comprises at least about 1,000 CFU of the endophytic bacterium applied on the exterior surface of the seed, the formulation comprising a microbial stabilizer.

32. A method for conferring one or more fitness benefits to an agricultural plant comprising:
a. Providing a seed or seedling of the plant;
b. Contacting said seed or seedling with the formulation of claim 21; and
c. Allowing the seed or seedling to grow under conditions that permit the endophytic bacterium to colonize the plant.

33. The method of claim 32, wherein:
(a) the one or more of the fitness benefits are selected from the group consisting of increased germination, increased biomass, increased flowering time, increased plant biomass, increased fruit or grain yield, increased biomass of the fruit or cob, and increased drought tolerance;
(b) the one or more of the fitness benefits is selected from the group consisting of increased germination rate, shortened time before onset of flowering, increased stomatal conductance, increased photosynthetic rates, increased plant height, and increased drought tolerance as compared to an agricultural plant grown under the same conditions;
(c) the one or more of the fitness benefits is selected from the group consisting of increased biomass, increased root biomass, increased leaf biomass, increased fruit or grain yield, and increased biomass of the fruit or cob as compared to an agricultural plant that was grown under stress-free conditions;
(d) the seed is allowed to grow under abiotic-stress free conditions and the one or more of the fitness benefits is selected from the group consisting of increased germination rate, increased biomass, shortened time before onset of flowering, increased plant biomass, increased fruit or grain yield, increased biomass of the fruit or cob, increased stomatal conductance, increased photosynthetic rates, increased plant height, and increased drought tolerance compared to a reference agricultural plant grown under the same conditions;
(e) the benefit is selected from the group consisting of: increased root biomass, increased root length, increased height, increased shoot length, increased leaf number, increased water use efficiency, increased tolerance to low nitrogen stress, increased nitrogen use efficiency, increased overall biomass, increase grain yield, increased photosynthesis rate, increased tolerance to drought, increased heat tolerance, increased salt tolerance, increased resistance to nematode stress, increased resistance to a fungal pathogen, increased resistance to a bacterial pathogen, increased resistance to a viral pathogen, a detectable modulation in the level of a metabolite, and a detectable modulation in the proteome, relative to reference seeds or agricultural plants derived from reference seeds;
(f) the benefit comprises at least two benefits selected from the group consisting of: increased root biomass, increased root length, increased height, increased shoot length, increased leaf number, increased water use efficiency, increased tolerance to low nitrogen stress, increased nitrogen use efficiency, increased overall biomass, increase grain yield, increased photosynthesis rate, increased tolerance to drought, increased heat tolerance, increased salt tolerance, increased resistance to nematode stress, increased resistance to a fungal pathogen, increased resistance to a bacterial pathogen, increased resistance to a viral pathogen, a detectable modulation in the level of a metabolite, and a detectable modulation in the proteome, relative to reference seeds or plants derived from reference seeds;
(g) the benefit is increased tolerance to low nitrogen stress or increased nitrogen use efficiency, and the endophytic bacterium is non-diazotrophic; or
(h) the benefit is increased tolerance to low nitrogen stress or increased nitrogen use efficiency, and the endophytic bacterium is diazotrophic.

34. A method of obtaining greater flowering uniformity in a population of agricultural monocot plants comprising:
(a) providing a population of monocot seeds;
(b) applying to an exterior surfaces of the population of seeds the formulation of claim 21; and
(c) allowing the population of seeds to grow at least to the flowering stage, wherein the population of plants has a reduction in the standard deviation in the flowering time of at least 5% when compared with a population of reference agricultural plants grown under the same conditions.

35. A method for treating seedlings, the method comprising:
(a) contacting foliage or the rhizosphere of a plurality of agricultural plant seedlings with a seed the formulation of claim 21; and
(b) growing the contacted seedlings.

36. The method of claim 35, wherein the contacting comprises spraying, immersing, coating, encapsulating, or dusting the seeds or seedlings with the formulation.

37. A method for modulating a plant trait comprising applying to (i) vegetation or an area adjacent the vegetation, (ii) soil, (iii) a seed the formulation of claim 21, wherein the formulation is capable of providing a benefit to (i) the vegetation, or to a crop produced from the vegetation, (ii) to seeds planted within the soil, or to a crop produced from plants grown in the soil.

38. The method of claim 37, wherein the vegetation is dicot seedlings or monocot seedlings.

39. The method of claim 28, wherein:
(a) the endophytic bacterium is capable of exhibiting production of an auxin, nitrogen fixation, production of an antimicrobial, production of a siderophore, mineral phosphate solubilization, production of a cellulase, production of a chitinase, production of a xylanase, or production of acetoin;
(b) the bacterial endophyte exhibits at least two of: production of an auxin, nitrogen fixation, production of an antimicrobial, production of a siderophore, mineral phosphate solubilization, production of a cellulase, production of a chitinase, production of a xylanase, and production of acetoin;
(c) the endophytic bacterium is capable of utilizing arabinose as a substrate; and/or
(d) the bacterial endophyte is present at a concentration of at least 10\hat{ }2 CFU/seed on the surface of the seeds after contacting.

40. A synthetic combination comprising a purified bacterial population in association with a plurality of seeds or seedlings of an agricultural plant, wherein the purified bacterial population comprises a first bacterial endophyte capable of at least one of: production of an auxin, nitrogen fixation, production of an antimicrobial, production of a siderophore, mineral phosphate solubilization, production of a cellulase, production of a chitinase, production of a xylanase, utilization of arabinose as a carbon source, and production of acetoin, or a combination of two or more thereof, wherein the first bacterial endophyte comprises a 16S rRNA nucleic acid sequence at least 95% identical to a nucleic acid sequence selected from the group consisting of SEQ ID NOs: 1-10, and wherein the bacterial endophyte is present in the synthetic combination in an amount effective to provide a benefit to the seeds or seedlings or the plants derived from the seeds or seedlings.

41. The synthetic combination of claim 40, wherein:
(a) the purified bacterial population is localized on the surface of the seeds or seedlings;
(b) the first bacterial endophyte is obtained from a plant species other than the seeds or seedlings of the synthetic combination;
(c) the first bacterial endophyte is obtained from a plant cultivar different from the cultivar of the seeds or seedlings of the synthetic combination;
(d) the first bacterial endophyte is obtained from a plant cultivar that is the same as the cultivar of the seeds or seedlings of the synthetic combination;
(e) the bacterial population comprises two or more families of bacterial endophytes;
(f) the bacterial population further comprises a second bacterial endophyte having an 16S nucleic acid sequence less than 95% identical to that of the first bacterial endophyte;
(g) the bacterial population further comprises a second bacterial endophyte, wherein the first and second bacterial endophytes are independently capable of at least one of production of an auxin, nitrogen fixation, production of an antimicrobial, production of a siderophore, mineral phosphate solubilization, production of a cellulase, production of a chitinase, production of a xylanase, utilization of arabinose as a carbon source, or production of acetoin, or a combination of two or more thereof;
(h) the bacterial population further comprises a second bacterial endophyte, wherein the first and second bacterial endophytes are capable of synergistically increasing at least one of: production of an auxin, nitrogen fixation, production of an antimicrobial, production of a siderophore, mineral phosphate solubilization, production of a cellulase, production of a chitinase, production of a xylanase, utilization of arabinose as a carbon source, or production of acetoin, or a combination of two or more thereof, in an amount effective to increase tolerance to drought relative to a reference plant;
(i) the first bacterial endophyte is capable of at least two of: production of an auxin, nitrogen fixation, production of an antimicrobial, production of a siderophore, mineral phosphate solubilization, production of a cellulase, production of a chitinase, production of a xylanase, utilization of arabinose as a carbon source, and production of acetoin;
(j) the benefit is selected from the group consisting of increased root biomass, increased root length, increased height, increased shoot length, increased leaf number, increased water use efficiency, increased overall biomass, increase grain yield, increased photosynthesis rate, increased tolerance to drought, increased heat tolerance, increased salt tolerance, increased resistance to nematode stress, increased resistance to a fungal pathogen, increased resistance to a bacterial pathogen, increased resistance to a viral pathogen, a detectable modulation in the level of a metabolite, and a detectable modulation in the proteome relative to a reference plant;
(k) the benefit comprises at least two benefits selected from the group consisting of increased root biomass, increased root length, increased height, increased shoot length, increased leaf number, increased water use efficiency, increased tolerance to low nitrogen stress, increased nitrogen use efficiency, increased overall biomass, increase grain yield, increased photosynthesis rate, increased tolerance to drought, increased heat tolerance, increased salt tolerance, increased resistance to nematode stress, increased resistance to a fungal pathogen, increased resistance to a bacterial pathogen, increased resistance to a viral pathogen, a detectable modulation in the level of a metabolite, and a detectable modulation in the proteome, relative to a reference plant;
(l) the combination comprises seeds and the first bacterial endophyte is associated with the seeds as a coating on the surface of the seeds;
(m) the combination comprises seedlings and the first bacterial endophyte is contacted with the seedlings as a spray applied to one or more leaves and/or one or more roots of the seedlings;
(n) the synthetic combination further comprises one or more additional bacterial endophyte species;
(o) the effective amount is at least 1×10\hat{ }CFU/per seed; and/or
(p) the combination comprises seeds and the effective amount is from about 1×10\hat{ }2 CFU/per seed to about 1×10\hat{ }8 CFU/per seed.

42. An agricultural product comprising a 1000 seed weight amount of the synthetic combination of claim 40, wherein the combination is produced by the step of contacting a plurality of agricultural plant seeds with a liquid formulation comprising a bacterial population at a concentration of at least 1 CFU per agricultural plant seed, wherein at least 10% of the CFUs present in the formulation are one or more bacterial endophytes, wherein the purified bacterial population comprises a first bacterial endophyte capable of at least one of: production of an auxin, nitrogen fixation, production of an antimicrobial, production of a siderophore, mineral phosphate solubilization, production of a cellulase, production of a chitinase, production of a xylanase, utilization of arabinose as a carbon source, and production of acetoin, or a combination of two or more thereof, wherein the first bacterial endophyte comprises a 16S rRNA nucleic acid sequence at least 95% identical to a nucleic acid sequence selected from the group consisting of SEQ ID NOs: 1-10, under conditions such that the formulation is associated with the surface of the seeds in a manner effective for the bacterial endophytes to confer a benefit to the seeds or to a crop comprising a plurality of agricultural plants produced from the seeds.

43. The agricultural product of claim 42, wherein:
(a) the bacterial endophytes are present in a concentration of from about 10\hat{ }2 to about 10\hat{ }5 CFU/ml; or
(b) the bacterial endophytes are present in a concentration is from about 10\hat{ }5 to about 10\hat{ }8 CFU/seed.
